(19) United States
(12) Patent Application Publication
(10) Pub. No.: US 2024/0174620 A1
(43) Pub. Date: May 30, 2024

(54) SOLID FORMS OF SAFLUFENACIL-SODIUM AND SAFLUFENACIL-POTASSIUM, PROCESS OF PREPARATION AND USE THEREOF

(71) Applicant: ADAMA AGAN LTD., Ashdod (IL)

(72) Inventors: Michael GRABARNICK, Meitar (IL); Sergio NAHMOUD, Ashdod (IL); Daniel MELIKER, Ariel (IL); Lior ZISERMAN, Holon (IL); Ashok Kumar JHA, Hyderabad (IN); Revanappa Vasantrao GALGE, Udgir (IN)

(21) Appl. No.: 18/280,120

(22) PCT Filed: Mar. 4, 2022
(86) PCT No.: PCT/IL2022/050246

§ 371 Date: Sep. 1, 2023

(30) Foreign Application Priority Data
Mar. 4, 2021 (IN) 202131009160

(51) Int. Cl. C07D 239/54; A01N 43/54; A01P 13/00

(52) U.S. Cl. CPC C07D 239/54; A01N 43/54; A01P 13/00

(57) ABSTRACT

The present invention relates to novel solid forms of saflufenacil-sodium or saflufenacil-potassium. The present invention further relates to a process of making said novel solid forms of saflufenacil-sodium or saflufenacil-potassium. Still further, the present invention relates to the use of the novel solid forms of saflufenacil-sodium or saflufenacil-potassium.

### RELATED APPLICATION/S

This application claims the benefit of priority of Indian Application No. 202131009160 filed on 4 Mar. 2021, the contents of which are incorporated herein by reference in their entirety.

### TECHNICAL FIELD OF THE INVENTION

The present invention relates to novel saflufenacil sodium and saflufenacil potassium compounds and solid forms of saflufenacil-sodium and saflufenacil-potassium. The present invention further relates to a process of making said novel solid forms of saflufenacil-sodium and saflufenacil-potassium. Still further, the present invention relates to the use of the novel solid forms of saflufenacil-sodium and saflufenacil-potassium.

### BACKGROUND OF THE INVENTION

Saflufenacil, having the chemical name 2-chloro-5-[3,6-dihydro-3-methyl-2,6-dioxo-4-(trifluoromethyl)-1(2H-pyrimidinyl]-4-fluoro-N-[[methyl(1-methylethyl)amino]sulfonyl]benzamide, has the following structural formula (I):

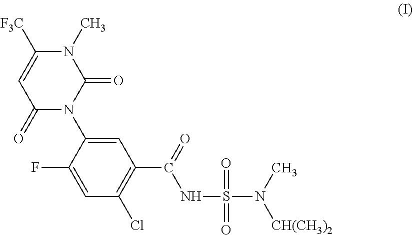 [SMILES: CC(C)N(C)S(=O)(=O)NC(=O)c1cc(-n2c(=O)cc(C(F)(F)F)n(C)c2=O)c(F)cc1Cl]

Saflufenacil belongs to the pyrimidindione and/or phenyluracil chemical groups, which is used as a herbicide, in particular as a foliar contact and residual broad-leaved weed herbicide. It is absorbed by foliage and roots, with translocation in the apoplast and limited movement in the phloem. Saflufenacil is used for foliar and residual control of broad-leaved weeds, including glyphosate- and ALS-resistant biotypes. Saflufenacil is an inhibitor of protoporphyrinogen oxidase and is applied pre-emergence in corn and sorghum, at 50-125 g/ha; is applied ore-plant for rapid foliar burn-down in soy beans, cereals, cotton, legumes, and post-directed in tree fruit and nuts, at 18-25 g/ha.

Saflufenacil is disclosed in WO 2001/083459. Further processes for its preparation are disclosed in WO 2003/097589, WO 2005/054208 and WO 2006/097589 and the earlier international application PCT/EP2006/062414, as well as U.S. Pat. No. 8,362,026, each of which is herein incorporated by reference in its entirety.

For example, U.S. Pat. No. 8,362,026 disclosed the following methods for preparing Saflufenacil as shown in Scheme 1 below:

1) reaction of an acid chloride with a sulfonamide; or

2) methylation reaction.

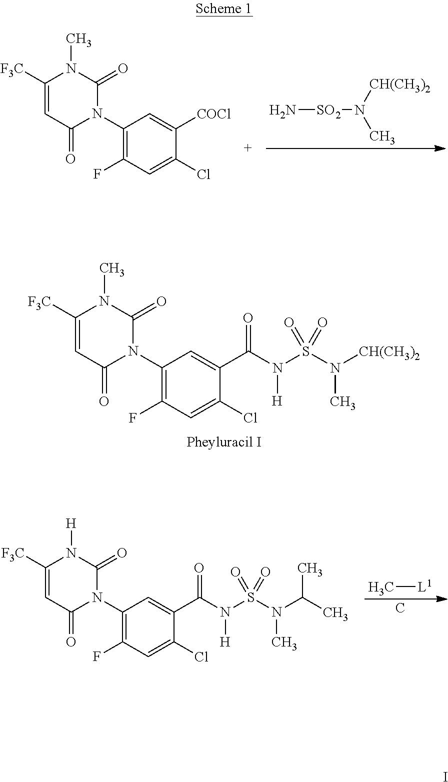

As noted in U.S. Pat. No. 8,362,026, Saflufenacil prepared by these aforementioned processes is amorphous and is extremally difficult to formulate. It is barely soluble in various liquid media, thus making it a challenge to create a stable liquid formulation. In particular, Saflufenacil has the tendency to precipitate from most solvents, organic or aqueous media; the solubility of Saflufenacil in water at pH 5 is 0.0025 g/100 mL and at pH 7 is 0.21 g/100 mL, both at 20° C. In acetonitrile, the solubility of Saflufenacil is 19.4 g/100 mL at 20° C. In acetone, the solubility of Saflufenacil is 27.5 g/100 mL at 20° C. In ethyl acetate, the solubility of Saflufenacil is 6.55 g/100 mL at 20° C. In tetrahydrofurane, the solubility of Saflufenacil is 36.2 g/100 mL at 20° C. In methanol, the solubility of Saflufenacil is 2.98 g/100 mL at 20° C. In isopropyl alcohol, the solubility of Saflufenacil is 0.25 g/100 mL at 20° C. In toluene, the solubility of Saflufenacil is 0.23 g/100 mL at 20° C. In 1-octanol, the solubility of Saflufenacil is <0.01 g/100 mL at 20° C. In n-heptane, the solubility of Saflufenacil is <0.005 g/100 mL at 20° C.

Furthermore, the two crystalline modifications of Saflufenacil, known in the art, Saflufenacil form II and crystalline form of saflufenacil hydrate, disclosed in WO 2008/043835 and WO 2008/043836, each of which is herein incorporated by reference in its entirety, are poorly soluble in aqueous media which can cause a problem for formulations.

Therefore, there is a need in the art for novel forms of Saflufenacil that exhibit improved properties, for example, an improved solubility in water.

Furthermore, as it was presented in U.S. Pat. No. 8,252,925, herein incorporated by reference in its entirety, methylation with dimethyl sulfate give mixture of the products difficult for separation and yield after chromatographic purification is on the level of 59% only. One of the main by-products of the methylation is dimethylated compound Ia;

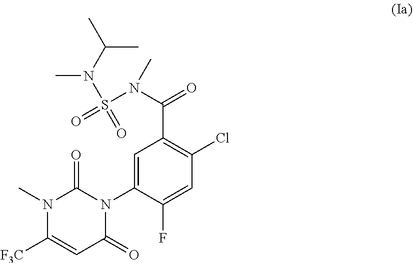 [SMILES: CC(C)N(C)S(=O)(=O)N(C)C(=O)c1cc(-n2c(=O)cc(C(F)(F)F)n(C)c2=O)c(F)cc1Cl]

The structure and most of the properties of this impurity are very close to the structure and properties of Saflufenacil. For this reason, purification of Saflufenacil from this impurity by a regular crystallization process, acceptable on the industrial scale, is very problematic and results in relatively low yield.

Therefore, there also is a need in the art for new processes for Saflufenacil purification that overcome shortcomings of previously known processes.

### SUMMARY OF THE INVENTION

The present invention provides saflufenacil-sodium and saflufenacil-potassium compounds. The present invention also provides solid forms of saflufenacil-sodium and saflufenacil-potassium. In embodiments, the solid form is an anhydrous form, a crystalline form, a hydrate form, a solvate form.

The present invention also provides processes of preparing solid forms of a saflufenacil-sodium or solid forms of a saflufenacil-potassium, comprising:

i. providing a solution of saflufenacil in an organic solvent;

ii. adding a base;

iii. optionally, heating;

iv. optionally, adding an anti-solvent;

v. optionally, cooling.

The present invention also provides an herbicidal composition comprising one or more saflufenacil-sodium or saflufenacil-potassium or Saflufenacil-sodium or Saflufenacil-potassium solid forms of Saflufenacil.

The present invention also provides an herbicidal composition comprising a Saflufenacil-sodium or Saflufenacil-potassium or Saflufenacil-sodium or Saflufenacil-potassium solid forms of Saflufenacil, and further comprising one or more additional herbicides.

The present invention also provides a method of controlling harmful weeds in a field of useful crops, the method comprising applying to the field a Saflufenacil-sodium or Saflufenacil-potassium or Saflufenacil-sodium or Saflufenacil-potassium solid forms of Saflufenacil.

The present invention also provides use of a Saflufenacil-sodium or Saflufenacil-potassium or Saflufenacil-sodium or Saflufenacil-potassium solid forms of Saflufenacil in the control of a harmful weed.

The present invention also provides an herbicidal composition comprising one or more of a Saflufenacil-sodium or Saflufenacil-potassium or solid form of Saflufenacil-sodium or Saflufenacil-potassium and at least one additional pesticide.

The present invention also provides a method for purification of Saflufenacil using Saflufenacil-sodium or Saflufenacil-potassium.

### BRIEF DESCRIPTION OF THE SEVERAL VIEWS OF THE DRAWINGS

FIG. 1. X-ray powder diffraction diffractogram of saflufenacil-sodium prepared from chlorobenzene (40 g preparation): SNa1.

FIG. 2. X-ray powder diffraction diffractogram of saflufenacil-sodium prepared from chlorobenzene (XRD pattern after two months storage at room temperature in glass bottle): SNa1a.

FIG. 3. X-ray powder diffraction diffractogram of saflufenacil-sodium prepared from 2-methyl tetrahydrofurane (40 g preparation): SNa2.

FIG. 4. X-ray powder diffraction diffractogram of saflufenacil-sodium prepared from 2-methyl tetrahydrofurane (XRD pattern after two months storage at room temperature in glass bottle): SNa2a.

FIG. 5. X-ray powder diffraction diffractogram of saflufenacil-potassium: SK.

FIG. 6. X-ray powder diffraction diffractogram of saflufenacil-sodium prepared from chlorobenzene (80 g preparation): SNa1b.

FIG. 7. X-ray powder diffraction diffractogram of saflufenacil-sodium Form prepared from 2-methyl tetrahydrofurane (80 g preparation): SNa2b.

FIG. 8. X-ray powder diffraction diffractogram of saflufenacil-sodium prepared from chlorobenzene (10 Kg preparation): SNa1c.

FIG. 9. X-ray powder diffraction diffractogram of saflufenacil-sodium prepared from chlorobenzene (10 Kg preparation), kept at 2-8° C. 1 week: SNa1d.

FIG. 10. X-ray powder diffraction diffractogram of saflufenacil-sodium prepared from chlorobenzene different volumes of MeOH/Chlorobenzene mixture and MeOH/2-Methyltetrahydrofuran mixture: SNa3.

FIG. 11.—X-ray powder diffraction diffractogram of saflufenacil-sodium prepared from different solvents: SNa4.

FIG. 12.—X-ray powder diffraction diffractogram of saflufenacil-sodium prepared from different solvents: SNa5.

FIG. 13.—X-ray powder diffraction diffractogram of saflufenacil-sodium prepared from Methyl isopropyl ketone: SNa6.

FIG. 14.—X-ray powder diffraction diffractogram of saflufenacil-sodium prepared from different solvents: SNa7.

FIG. 15.—X-ray powder diffraction diffractogram of saflufenacil-sodium prepared from ethanol: SNa8.

### DETAILED DESCRIPTION OF THE INVENTION

Aspects and embodiments of the present invention will now be described. Novel solid forms of Saflufenacil-sodium and Saflufenacil-potassium have now been found, which when formulated into agrochemical compositions, exhibit a higher degree of solubility in the farmer's tank-mix. Therefore, problematic issues of active ingredient dispersion in tank-mix (such as sedimentations, nozzle clogging and so forte) can be overcome at the recommended usage concentrations.

It was surprisingly discovered that the new compounds Saflufenacil-sodium and Saflufenacil-potassium have strongly different physical properties from compound Ia, especially solubility both in organic solvents and in water, and may be easily used for Saflufenacil purification from compound Ia.

In an embodiment, the present invention provides a purification process of Saflufenacil using solid forms of Saflufenacil-sodium or Saflufenacil-potassium in an efficient, high yield and green synthetic process including, preparing Saflufenacil by methylation reaction of the Saflufenacil immediate precursor of the formula Ib;

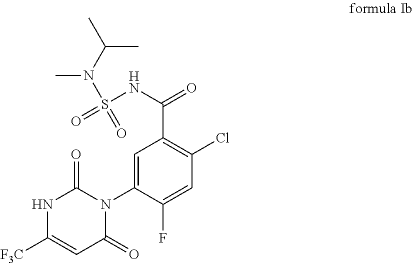 [SMILES: CC(C)N(C)S(=O)(=O)NC(=O)c1cc(-n2c(=O)cc(C(F)(F)F)[nH]c2=O)c(F)cc1Cl]

crude Saflufenacil than converted to the Saflufenacil-sodium or Saflufenacil-potassium solid form, allowing most of the organic impurities, including the dimethylated by-product of the formula Ia, to be dissolved in the organic solvent of the reaction and therefore, easily separated from the filtered solid. The resulting clean and stable solid form of Saflufenacil-sodium or Saflufenacil-potassium then easily may be used to obtain purified Saflufenacil by acidification.

In one aspect, the present invention relates to a Saflufenacil-sodium or Saflufenacil-potassium.

Saflufenacil (2-chloro-5-[3,6-dihydro-3-methyl-2,6-dioxo-4-(trifluoromethyl)-1(2H-pyrimidinyl]-4-fluoro-N-[[methyl(1-methylethyl)amino]sulfonyl]benzamide) has the following structural formula (I):

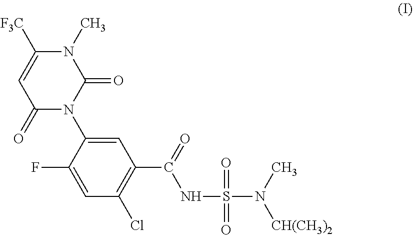 [SMILES: CC(C)N(C)S(=O)(=O)NC(=O)c1cc(-n2c(=O)cc(C(F)(F)F)n(C)c2=O)c(F)cc1Cl]

In one embodiment, the present invention provides a Saflufenacil-sodium or Saflufenacil-potassium, wherein the Saflufenacil-sodium is a salt.

In one embodiment, the present invention provides a Saflufenacil-sodium or saflufenacil-potassium wherein the Saflufenacil-potassium is a salt.

In one embodiment, the present invention provides a Saflufenacil-sodium or Saflufenacil-potassium, wherein the Saflufenacil-sodium is a salt or the Saflufenacil-potassium is a salt.

In one embodiment, the present invention provides a salt, wherein the salt is a sodium salt of Saflufenacil.

In one embodiment, the present invention provides a salt, wherein the salt is a potassium salt of Saflufenacil.

In one embodiment, the present invention provides a salt, wherein the salt is a sodium or potassium salt of Saflufenacil.

In one aspect, the present invention relates to a solid form of a Saflufenacil-sodium or Saflufenacil-potassium.

In one embodiment, the present invention provides a solid form of the Saflufenacil-sodium.

In one embodiment, the present invention provides a solid form of the Saflufenacil-potassium.

In one embodiment, the present invention provides a solid form of the Saflufenacil-sodium, wherein the solid form is an anhydrous form, a crystalline form, a hydrate form, a solvate form, a polymorph form, a crystalline form with low crystallinity, or a crystalline form with high crystallinity.

In one embodiment, the present invention provides a solid form of the Saflufenacil-potassium, wherein the solid is an anhydrous form, a crystalline form, a hydrate form, a solvate form, a polymorph form, a crystalline form with low crystallinity, or a crystalline form with high crystallinity.

In one embodiment, the present invention provides a solid form of the Saflufenacil-sodium or Saflufenacil-potassium, wherein the solid form is an anhydrous form, a crystalline form, a hydrate form, a solvate form, a polymorph form, a crystalline form with low crystallinity or a crystalline form with high crystallinity, or any combination thereof.

In one embodiment, the present invention provides a solid form of the Saflufenacil-sodium (SNa1), wherein the solid form exhibits at least 3 of the following peaks expressed as degrees 2θ values in an X-ray diffractogram recorded using Cu—K_{α} radiation at 25° C.:
peak (1): 2θ=5.9±0.3
peak (2): 2θ=14.7±0.2
peak (3): 2θ=18.6±0.2
peak (4): 2θ=19.5±0.2
peak (5): 2θ=22.7±0.4.

In one embodiment, the present invention provides a solid form of Saflufenacil-sodium (SNa1), wherein the solid form exhibits at least 3 of the following peaks expressed as degrees 2θ values in an X-ray diffractogram recorded using Cu—K_{α} radiation at 25° C.:
peak (1): 2θ=6.1±0.2
peak (2): 2θ=14.9±0.2
peak (3): 2θ=18.5±0.2
peak (4): 2θ=19.7±0.2
peak (5): 2θ=23.3±0.2.

In one embodiment, the present invention provides a solid form of Saflufenacil-sodium (SNa1), wherein at least 4 of the peaks (1) to (5) are exhibited.

In one embodiment, the present invention provides a solid form of Saflufenacil-sodium (SNa1), wherein all the peaks (1) to (5) are exhibited.

In one embodiment, the present invention provides a solid form of the Saflufenacil-sodium (SNa1), wherein the solid form exhibits at least 3 of the following peaks expressed as degrees 2θ values in an X-ray diffractogram recorded using Cu—K_{α} radiation at 25° C.:
peak (1): 2θ=5.9±0.3
peak (2): 2θ=14.7±0.2
peak (3): 2θ=18.6±0.2
peak (4): 2θ=19.5±0.2
peak (5): 2θ=22.7±0.4
   or
   the solid form of the Saflufenacil-sodium (SNa1), has a secondary pick list, wherein the solid form exhibits at least 3 of the following peaks expressed as degrees 2θ values in an X-ray diffractogram recorded using Cu—K_{α} radiation at 25° C.:
peak (1): 2θ=6.1±0.2
peak (2): 2θ=14.9±0.2
peak (3): 2θ=18.5±0.2
peak (4): 2θ=19.7±0.2
peak (5): 2θ=23.3±0.2.

In particular such embodiment, the present invention provides a solid form of Saflufenacil-sodium (SNa1a), wherein the solid form exhibits at least 2 of the following peaks expressed as degrees 2θ values in an X-ray diffractogram recorded using Cu—K_{α} radiation at 25° C.:
peak (1): 2θ=4.3±0.2
peak (2): 2θ=16.4±0.2
peak (3): 2θ=19.6±0.2
peak (4): 2θ=22.5±0.2.

In one embodiment, the present invention provides a solid form of Saflufenacil-sodium (SNa1a), wherein at least 3 of the peaks (1) to (4) are exhibited.

In one embodiment, the present invention provides a solid form of Saflufenacil-sodium (SNa1a), wherein all the peaks (1) to (4) are exhibited.

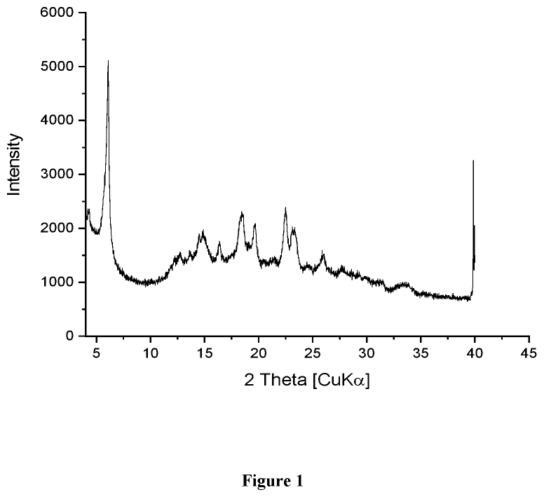

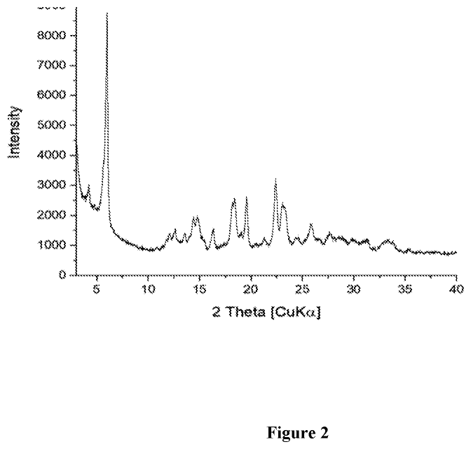

In one embodiment, the present invention provides a solid form of Saflufenacil-sodium (SNa1 or SNa1a) having an X-ray diffractogram pattern substantially as shown in FIG. 1 or 2.

In one embodiment, the present invention provides a solid form of Saflufenacil-sodium (SNa1b), wherein the solid form exhibits at least 3 of the following peaks expressed as degrees 2θ values in an X-ray diffractogram recorded using Cu—K_{α} radiation at 25° C.:
peak (1): 2θ=6.1±0.2
peak (2): 2θ=14.7±0.2
peak (3): 2θ=18.3±0.2
peak (4): 2θ=19.5±0.2
peak (5): 2θ=23.1±0.2.

In one embodiment, the present invention provides a solid form of Saflufenacil-sodium (SNa1b), wherein at least 4 of the peaks (1) to (5) are exhibited.

In one embodiment, the present invention provides a solid form of Saflufenacil-sodium (SNa1b), wherein all the peaks (1) to (5) are exhibited.

In particular such embodiment, the present invention provides a solid form of Saflufenacil-sodium (SNa1b), wherein the solid form exhibits at least 2 of the following peaks expressed as degrees 2θ alues in an X-ray diffractogram recorded using Cu—K_{α} radiation at 25° C.:
peak (1): 2θ=16.3±0.2
peak (2): 2θ=19.5±0.2
peak (3): 2θ=22.5±0.2.

In one embodiment, the present invention provides a solid form of Saflufenacil-sodium (SNa1b), wherein all the peaks (1) to (3) are exhibited.

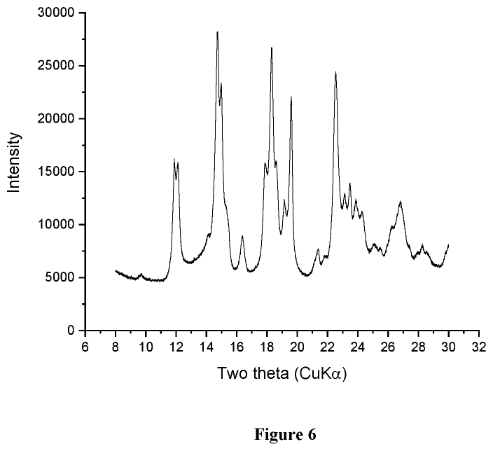

In one embodiment, the present invention provides a solid form of Saflufenacil-sodium (SNa1b) having an X-ray diffractogram pattern substantially as shown in FIG. 6.

In one embodiment, the present invention provides a solid form of the Saflufenacil-sodium (SNa1b), wherein the solid form exhibits at least 3 of the following peaks expressed as degrees 2θ values in an X-ray diffractogram recorded using Cu—K_{α} radiation at 25° C.:
peak (1): 2θ=6.1 ±0.2
peak (2): 2θ=14.7±0.2
peak (3): 2θ=18.3±0.2
peak (4): 2θ=19.5±0.2
peak (5): 2θ=23.1±0.2
   or
   the solid form of the Saflufenacil-sodium (SNa1b), has a secondary pick list, wherein the solid form exhibits at least 2 of the following peaks expressed as degrees 2θ values in an X-ray diffractogram recorded using Cu—K_{α} radiation at 25° C.:
peak (1): 2θ=16.3±0.2
peak (2): 2θ=19.5±0.2
peak (3): 2θ=22.5±0.2.

In one embodiment, the present invention provides a solid form of the Saflufenacil-sodium (SNa1b), wherein at least 4 of the peaks (1) to (5) are exhibited.

In one embodiment, the present invention provides a solid form of the Saflufenacil-sodium (SNa1b), wherein all the peaks (1) to (5) the peaks (1) to (3) are exhibited.

In one embodiment, the present invention provides a solid form of Saflufenacil-sodium (SNa2), wherein the solid form exhibits at least 3 of the following peaks expressed as degrees 2θ values in an X-ray diffractogram recorded using Cu—K_{α} radiation at 25° C.:
peak (1): 2θ=5.9±0.2
peak (2): 2θ=14.9±0.2
peak (3): 2θ=18.5±0.2
peak (4): 2θ=22.7±0.2.

In one embodiment, the present invention provides a solid form of Saflufenacil-sodium (SNa2), wherein at least 3 of the peaks (1) to (4) are exhibited.

In one embodiment, the present invention provides a solid form of Saflufenacil-sodium (SNa2), wherein all the peaks (1) to (4) are exhibited.

In one embodiment, the present invention provides a solid form of the Saflufenacil-sodium (SNa2), wherein the solid form exhibits at least 3 of the following peaks expressed as degrees 2θ values in an X-ray diffractogram recorded using Cu—K_{α} radiation at 25° C.:
peak (1): 2θ=5.9±0.2
peak (2): 2θ=14.9±0.2
peak (3): 2θ=18.5±0.2
peak (4): 2θ=22.7±0.2
   or
   the solid form of the Saflufenacil-sodium (SNa2a), has a secondary peak list, wherein the solid form exhibits at least 2 of the following peaks expressed as degrees 2θ values in an X-ray diffractogram recorded using Cu—K_{α} radiation at 25° C.:
peak (1): 2θ=4.9±0.2
peak (2): 2θ=12.3±0.2
peak (3): 2θ=16.3±0.2
peak (4): 2θ=19.5±0.2.

In one embodiment, the present invention provides a solid form of the Saflufenacil-sodium (SNa2), wherein at least 3 of the peaks (1) to (4) are exhibited.

In one embodiment, the present invention provides a solid form of the Saflufenacil-sodium (SNa2), wherein all the peaks (1) to (4) are exhibited.

In particular such embodiment, the present invention provides a solid form of Saflufenacil-sodium (SNa2a), wherein the solid form exhibits at least 2 of the following peaks expressed as degrees 2θ values in an X-ray diffractogram recorded using Cu—K_{α} radiation at 25° C.:
peak (1): 2θ=4.9±0.2
peak (2): 2θ=12.3±0.2
peak (3): 2θ=16.3±0.2
peak (4): 2θ=19.5±0.2.

In one embodiment, the present invention provides a solid form of Saflufenacil-sodium (SNa2a), wherein at least 3 of the peaks (1) to (4) are exhibited.

In one embodiment, the present invention provides a solid form of Saflufenacil-sodium (SNa2a), wherein all the peaks (1) to (4) are exhibited.

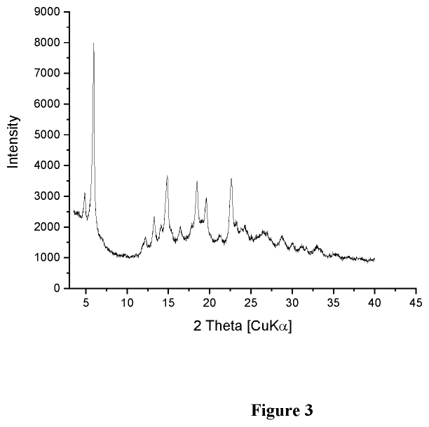

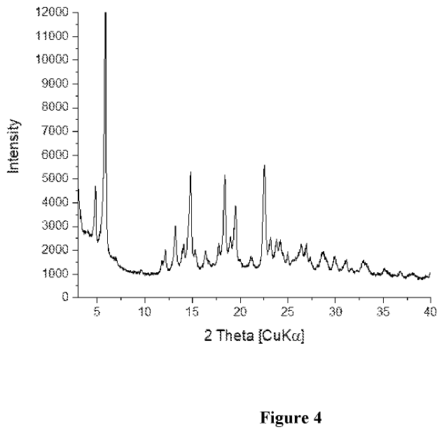

In one embodiment, the present invention provides a solid form of Saflufenacil-sodium (SNa2 or SNa2a) having an X-ray diffractogram pattern substantially as shown in FIG. 3 or 4.

In one embodiment, the present invention provides a solid form of Saflufenacil-sodium (SNa2b), wherein the solid form exhibits at least 3 of the following peaks expressed as degrees 2θ values in an X-ray diffractogram recorded using Cu—K_{α} radiation at 25° C.:
peak (1): 2θ=5.9±0.2
peak (2): 2θ=14.7±0.2
peak (3): 2θ=18.5±0.2
peak (4): 2θ=22.7±0.2.

In one embodiment, the present invention provides a solid form of Saflufenacil-sodium (SNa2b), wherein at least 3 of the peaks (1) to (4) are exhibited.

In one embodiment, the present invention provides a solid form of Saflufenacil-sodium (SNa2b), wherein all the peaks (1) to (4) are exhibited.

In particular such embodiment, the present invention provides a solid form of Saflufenacil-sodium (SNa2b), wherein the solid form exhibits at least 2 of the following peaks expressed as degrees 2θ values in an X-ray diffractogram recorded using Cu—K_{α} a radiation at 25° C.:
peak (1): 2θ=4.9±0.2
peak (2): 2θ=12.1±0.2
peak (3): 2θ=16.5±0.2
peak (4): 2θ=19.6±0.2.

In one embodiment, the present invention provides a solid form of Saflufenacil-sodium (SNa2b), wherein at least 3 of the peaks (1) to (4) are exhibited.

In one embodiment, the present invention provides a solid form of Saflufenacil-sodium (SNa2b), wherein all the peaks (1) to (4) are exhibited of Saflufenacil-sodium.

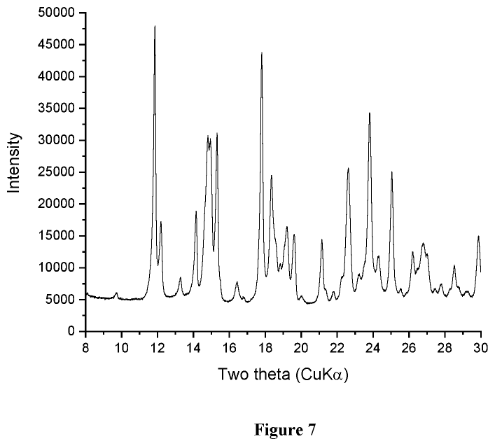

In one embodiment, the present invention provides a solid form of Saflufenacil-sodium (SNa2b) having an X-ray diffractogram pattern substantially as shown in FIG. 7.

In one embodiment, the present invention provides a solid form of the Saflufenacil-sodium (SNa2b), wherein the solid form exhibits at least 3 of the following peaks expressed as degrees 2θ values in an X-ray diffractogram recorded using Cu—K_{α} radiation at 25° C.:
peak (1): 2θ=5.9±0.2
peak (2): 2θ=14.7±0.2
peak (3): 2θ=18.5±0.2
peak (4): 2θ=22.7±0.2
   or
   the solid form of the Saflufenacil-sodium (SNa2b), has a secondary peak list, wherein the solid form exhibits at least 2 of the following peaks expressed as degrees 2θ values in an X-ray diffractogram recorded using Cu—K_{α} radiation at 25° C.:
peak (1): 2θ=4.9±0.2
peak(2): 2θ=12.1±0.2
peak (3): 2θ=16.5±0.2
peak (4): 2θ=19.6±0.2.

In one embodiment, the present invention provides a solid form of the Saflufenacil-sodium (SNa2b), wherein at least 3 of the peaks (1) to (4) are exhibited.

In one embodiment, the present invention provides a solid form of the Saflufenacil-sodium (SNa2b), wherein all the peaks (1) to (4) are exhibited.

In one embodiment, the present invention provides a solid form of Saflufenacil-sodium (SNa1c), wherein the solid form exhibits at least 3 of the following peaks expressed as degrees 2θ values in an X-ray diffractogram recorded using Cu—K_{α} radiation at 25° C.:
30 peak (1): 2θ=6.2±0.2
peak(2): 2θ=14.5±0.2
peak (3): 2θ=18.5±0.2
peak (4): 2θ=19.6±0.2
peak (5): 2θ=22.4±0.2.

In one embodiment, the present invention provides a solid form of Saflufenacil-sodium (SNa1c), wherein at least 3 of the peaks (1) to (5) are exhibited.

In one embodiment, the present invention provides a solid form of Saflufenacil-sodium (SNa1c), wherein all the peaks (1) to (5) are exhibited.

In one embodiment, the present invention provides a solid form of Saflufenacil-sodium (SNa1c), wherein the solid form exhibits at least 2 of the following peaks expressed as degrees 2θ values in an X-ray diffractogram recorded using Cu—K_{α} radiation at 25° C.:
peak (1): 2θ=11.0±0.2
peak(2): 2θ=11.4±0.2
peak (3): 2θ=11.8±0.2
peak (4): 2θ=13.7±0.2.

In one embodiment, the present invention provides a solid form of Saflufenacil-sodium (SNa1c), wherein at least 3 of the peaks (1) to (4) are exhibited.

In one embodiment, the present invention provides a solid form of Saflufenacil-sodium (SNa1c), wherein all the peaks (1) to (4) are exhibited.

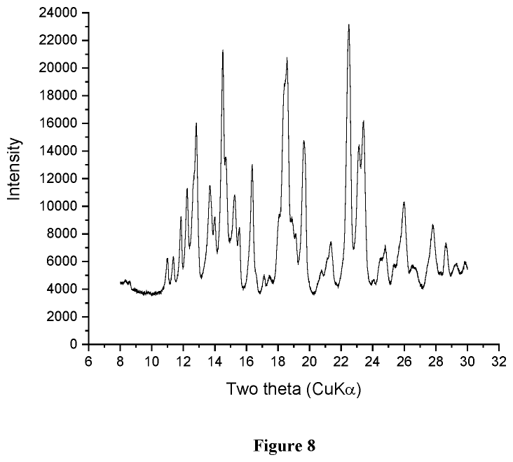

In one embodiment, the present invention provides a solid form of Saflufenacil-sodium (SNa1c) having an X-ray diffractogram pattern substantially as shown in FIG. 8.

In one embodiment, the present invention provides a solid form of any of the Saflufenacil-sodium (SNa1c), wherein the solid form exhibits at least 3 of the following peaks expressed as degrees 2θ values in an X-ray diffractogram recorded using Cu—K_{α} radiation at 25° C.:
peak (1): 2θ=6.2±0.2
peak(2): 2θ=14.5±0.2
peak (3): 2θ=18.5±0.2
peak (4): 2θ=19.6±0.2
peak (5): 2θ=22.4±0.2
   or
   the solid form of any of the Saflufenacil-sodium (SNa1c), has a secondary peak list, wherein the solid form exhibits at least 2 of the following peaks expressed as degrees 2θ values in an X-ray diffractogram recorded using Cu—K_{α} radiation at 25° C.:
peak(1): 2θ=11.0±0.2
peak (2): 2θ=11.4±0.2
peak (3): 2θ=11.8±0.2
peak (4): 2θ=13.7±0.2.

In one embodiment, the present invention provides a solid form of the Saflufenacil-sodium (SNa1c), wherein at least 3 of the peaks (1) to (5) or of the peaks (1) to (4) are exhibited.

In one embodiment, the present invention provides a solid form of the Saflufenacil-sodium (SNa1c), wherein all the peaks (1) to (5) or of the peaks (1) to (4) are exhibited.

In one embodiment, the present invention provides a solid form of the Saflufenacil-sodium (SNa3), wherein the solid form exhibits at least 2 of the following peaks expressed as degrees 2θ values in an X-ray diffractogram recorded using Cu—K_{α} radiation at 25° C.:
peak (1): 2θ=5.8±0.2
peak (2): 2θ=11.7±0.2
peak (3): 2θ=17.7±0.2
peak (4): 2θ=23.7±0.2.

In one embodiment, the present invention provides a solid form of the Saflufenacil-sodium (SNa3), wherein at least 3 of the peaks (1) to (4) are exhibited.

In one embodiment, the present invention provides a solid form of the Saflufenacil-sodium (SNa3), wherein all the peaks (1) to (4) are exhibited.

In one embodiment, the present invention provides a solid form of the Saflufenacil-sodium (SNa3), wherein the solid form exhibits at least 4 of the following peaks expressed as degrees 2θ values in an X-ray diffractogram recorded using Cu—K_{α} radiation at 25° C.:
peak (1): 2θ=5.8±0.2
peak (2): 2θ=11.7±0.2
peak (3): 2θ=15.2±0.2
peak (4): 2θ=17.7±0.2
peak (5): 2θ=21.0±0.2
peak (6): 2θ=23.7±0.2
peak (7): 2θ=24.9±0.2
peak (8): 2θ=29.8±0.2.

In one embodiment, the present invention provides a solid form of the Saflufenacil-sodium (SNa3), wherein at least 5 of the peaks (1) to (8) are exhibited.

In one embodiment, the present invention provides a solid form of the Saflufenacil-sodium (SNa3), wherein all the peaks (1) to (8) are exhibited.

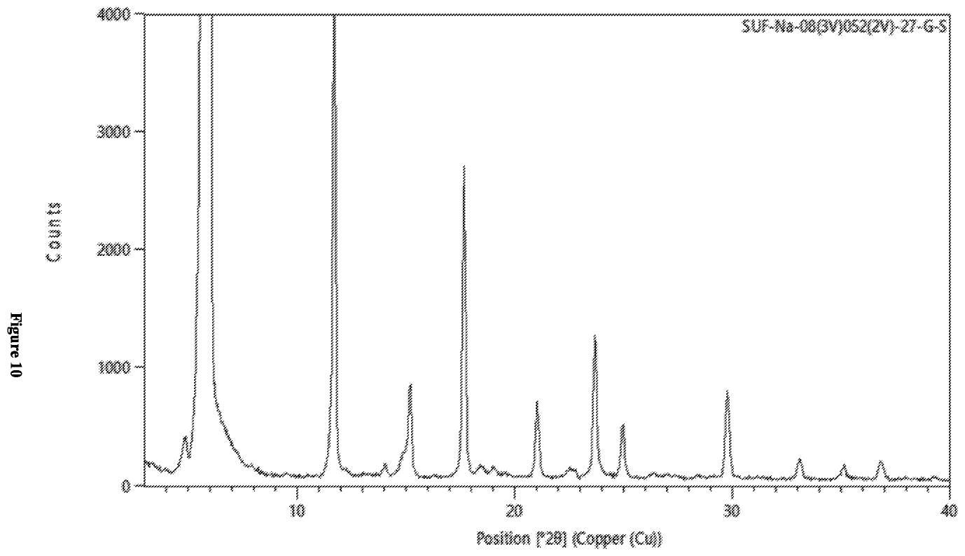

In one embodiment, the present invention provides a solid form of Saflufenacil-sodium (SNa3) having an X-ray diffractogram pattern substantially as shown in FIG. 10.

In one embodiment, the present invention provides a solid form of the Saflufenacil-sodium (SNa3), wherein the solid form exhibits at least 2 of the following peaks expressed as degrees 2θ values in an X-ray diffractogram recorded using Cu—K_{α} a radiation at 25° C.:
peak (1): 2θ=5.8±0.2
peak (2): 2θ=11.7±0.2
peak (3): 2θ=17.7±0.2
peak (4): 2θ=23.7±0.2
   or
   the solid form of the Saflufenacil-sodium (SNa3), has a secondary peak list, wherein the solid form exhibits at least 4 of the following peaks expressed as degrees 2θ values in an X-ray diffractogram recorded using Cu—K_{α} radiation at 25° C.:
peak (1): 2θ=5.8±0.2
peak (2): 2θ=11.7±0.2
peak (3): 2θ=15.2±0.2
peak (4): 2θ=17.7±0.2
peak (5): 2θ=21.0±0.2
peak (6): 2θ=23.7±0.2
peak (7): 2θ=24.9±0.2
peak (8): 2θ=29.8±0.2.

In one embodiment, the present invention provides a solid form of the Saflufenacil-sodium (SNa3), wherein at least 3 of the peaks (1) to (4) or at least 5 of the peaks (1) to (8) are exhibited.

In one embodiment, the present invention provides a solid form of the Saflufenacil-sodium (SNa3), wherein all the peaks (1) to (4) or of the peaks (1) to (8) are exhibited.

In one embodiment, the present invention provides a solid form of the Saflufenacil-sodium (SNa4), wherein the solid form exhibits at least 1 of the following peaks expressed as degrees 2θ values in an X-ray diffractogram recorded using Cu—K_{α} radiation at 25° C.:
peak (1): 2θ=6.7±0.2
peak (2): 2θ=13.4±0.2
peak (3): 2θ=20.1±0.2.

In one embodiment, the present invention provides a solid form of the Saflufenacil-sodium (SNa4), wherein at least 2 of the peaks (1) to (3) are exhibited.

In one embodiment, the present invention provides a solid form of the Saflufenacil-sodium (SNa4), wherein all the peaks (1) to (3) are exhibited.

In one embodiment, the present invention provides a solid form of the Saflufenacil-sodium (SNa4), wherein the solid form exhibits at least 4 of the following peaks expressed as degrees 2θ values in an X-ray diffractogram recorded using Cu—K_{α} radiation at 25° C.:
peak (1): 2θ=6.7±0.2
peak (2): 2θ=13.4±0.2
peak (3): 2θ=14.2±0.2
peak (4): 2θ=18.0±0.2
peak (5): 2θ=20.1±0.2
peak (6): 2θ=23.1±0.2
peak (7): 2θ=24.0±0.2
peak (8): 2θ=24.5±0.2.

In one embodiment, the present invention provides a solid form of the Saflufenacil-sodium (SNa4), wherein at least 5 of the peaks (1) to (8) are exhibited.

In one embodiment, the present invention provides a solid form of the Saflufenacil-sodium (SNa4), wherein all the peaks (1) to (8) are exhibited.

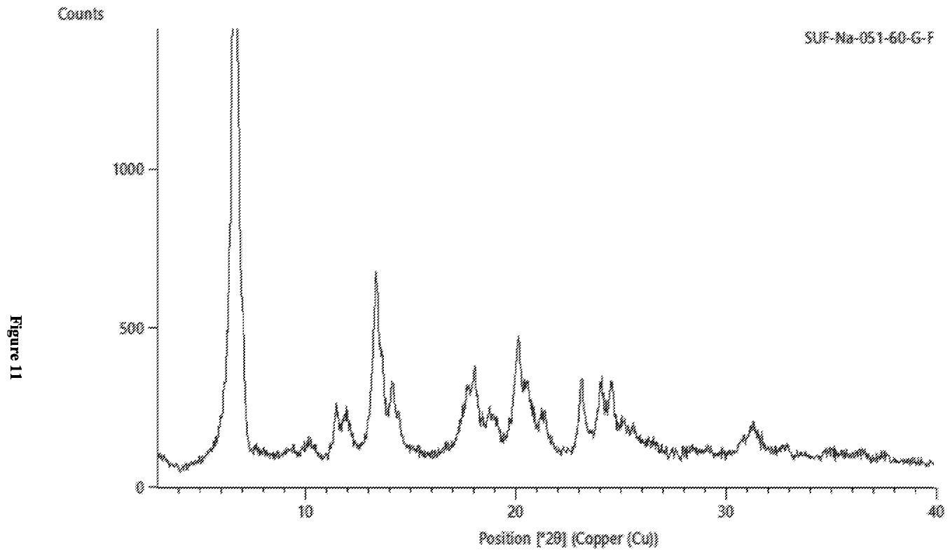

In one embodiment, the present invention provides a solid form of Saflufenacil-sodium (SNa4) having an X-ray diffractogram pattern substantially as shown in FIG. 11.

In one embodiment, the present invention provides a solid form of the Saflufenacil-sodium (SNa4), wherein the solid form exhibits at least 2 of the following peaks expressed as degrees 2θ values in an X-ray diffractogram recorded using Cu—K_{α} radiation at 25° C.:
peak (1): 2θ=6.7±0.2
peak (2): 2θ=13.4±0.2
peak (3): 2θ=20.1±0.2
   or
   the solid form of the Saflufenacil-sodium (SNa4), has a secondary peak list, wherein the solid form exhibits at least 4 of the following peaks expressed as degrees 2θ values in an X-ray diffractogram recorded using Cu—K_{α} radiation at 25° C.:
peak (1): 2θ=6.7±0.2
peak (2): 2θ=13.4±0.2
peak (3): 2θ=14.2±0.2
peak (4): 2θ=18.0±0.2
peak (5): 2θ=20.1±0.2
peak (6): 2θ=23.1±0.2
peak (7): 2θ=24.0±0.2
peak (8): 2θ=24.5±0.2.

In one embodiment, the present invention provides a solid form of the Saflufenacil-sodium (SNa4), wherein at least 5 of the peaks (1) to (8) are exhibited.

In one embodiment, the present invention provides a solid form of the Saflufenacil-sodium (SNa4), wherein all the peaks (1) to (3) or of the peaks (1) to (8) are exhibited.

In one embodiment, the present invention provides a solid form of the Saflufenacil-sodium (SNa5), wherein the solid form exhibits at least 3 of the following peaks expressed as degrees 2θ values in an X-ray diffractogram recorded using Cu—K_{α} radiation at 25° C.:
peak (1): 2θ=4.2±0.2
peak (2): 2θ=7.1±0.2
peak (3): 2θ=13.5±0.2
peak (4): 2θ=17.4±0.2
peak (5): 2θ=24.9±0.2
peak (6): 2θ=26.0±0.2.

In one embodiment, the present invention provides a solid form of the Saflufenacil-sodium (SNa5), wherein at least 4 of the peaks (1) to (6) are exhibited.

In one embodiment, the present invention provides a solid form of the Saflufenacil-sodium (SNa5), wherein all the peaks (1) to (6) are exhibited.

In one embodiment, the present invention provides a solid form of the Saflufenacil-sodium (SNa5), wherein the solid form exhibits at least 4 of the following peaks expressed as degrees 2θ values in an X-ray diffractogram recorded using Cu—K_{α} radiation at 25° C.:
peak (1): 2θ=4.2±0.2
peak (2): 2θ=7.1±0.2
peak (3): 2θ=9.8±0.2
peak (4): 2θ=13.5±0.2
peak (5): 2θ=14.3±0.2
peak (6): 2θ=17.4±0.2
peak (7): 2θ=21.5±0.2
peak (8): 2θ=24.9±0.2
peak (9): 2θ=26.0±0.2.

In one embodiment, the present invention provides a solid form of the Saflufenacil-sodium (SNa5), wherein at least 6 of the peaks (1) to (9) are exhibited.

In one embodiment, the present invention provides a solid form of the Saflufenacil-sodium (SNa5), wherein all the peaks (1) to (9) are exhibited.

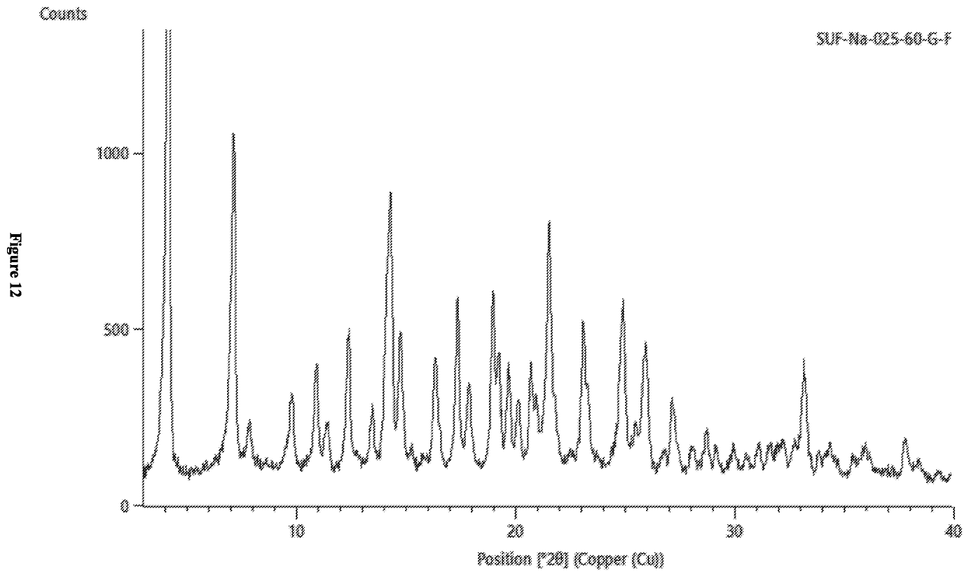

In one embodiment, the present invention provides a solid form of Saflufenacil-sodium (SNa5) having an X-ray diffractogram pattern substantially as shown in FIG. 12.

In one embodiment, the present invention provides a solid form of the Saflufenacil-sodium (SNa5), wherein the solid form exhibits at least 3 of the following peaks expressed as degrees 2θ values in an X-ray diffractogram recorded using Cu—K_{α} radiation at 25° C.:
peak (1): 2θ=4.2±0.2
peak (2): 2θ=7.1±0.2
peak (3): 2θ=13.5±0.2
peak (4): 2θ=17.4±0.2
peak (5): 2θ=24.9±0.2
peak (6): 2θ=26.0±0.2
   or
   the solid form of the Saflufenacil-sodium (SNa5), has a secondary peak list, wherein the solid form exhibits at least 4 of the following peaks expressed as degrees 2θ values in an X-ray diffractogram recorded using Cu—K_{α} radiation at 25° C.:
peak (1): 2θ=4.2±0.2
peak (2): 2θ=7.1±0.2
peak (3): 2θ=9.8±0.2
peak (4): 2θ=13.5±0.2
peak (5): 2θ=14.3±0.2
peak (6): 2θ=17.4±0.2
peak (7): 2θ=21.5±0.2
peak (8): 2θ=24.9±0.2
peak (9): 2θ=26.0±0.2.

In one embodiment, the present invention provides a solid form of the Saflufenacil-sodium (SNa5), wherein at least 4 of the peaks (1) to (6) or at least 6 of the peaks (1) to (9) are exhibited.

In one embodiment, the present invention provides a solid form of the Saflufenacil-sodium (SNa5), wherein all the peaks (1) to (6) or of the peaks (1) to (9) are exhibited.

In one embodiment, the present invention provides a solid form of the Saflufenacil-sodium (SNa6), wherein the solid form exhibits at least 3 of the following peaks expressed as degrees 2θ values in an X-ray diffractogram recorded using Cu—K_{α} radiation at 25° C.:
peak (1): 2θ=4.1±0.2
peak (2): 2θ=7.1±0.2
peak (3): 2θ=8.7±0.2
peak (4): 2θ=13.4±0.2
peak (5): 2θ=16.9±0.2
peak (6): 2θ=17.2±0.2
peak (7): 2θ=24.7±0.2.

In one embodiment, the present invention provides a solid form of the Saflufenacil-sodium (SNa6), wherein at least 4 of the peaks (1) to (7) are exhibited.

In one embodiment, the present invention provides a solid form of the Saflufenacil-sodium (SNa6), wherein all the peaks (1) to (7) are exhibited.

In one embodiment, the present invention provides a solid form of the Saflufenacil-sodium (SNa6), wherein the solid form exhibits at least 5 of the following peaks expressed as degrees 2θ values in an X-ray diffractogram recorded using Cu—K_{α} radiation at 25° C.:
peak (1): 2θ=4.1±0.2
peak (2): 2θ=7.1±0.2
peak (3): 2θ=8.7±0.2
peak (4): 2θ=9.7±0.2
peak (5): 2θ=11.4±0.2
peak (6): 2θ=13.4±0.2
peak (7): 2θ=16.9±0.2
peak (8): 2θ=17.2±0.2
peak (9): 2θ=19.0±0.2
peak (10): 2θ=21.4±0.2
peak (11): 2θ=22.5±0.2
peak (12): 2θ=24.7±0.2.

In one embodiment, the present invention provides a solid form of the Saflufenacil-sodium (SNa6), wherein at least 7 of the peaks (1) to (12) are exhibited.

In one embodiment, the present invention provides a solid form of the Saflufenacil-sodium (SNa6), wherein all the peaks (1) to (12) are exhibited.

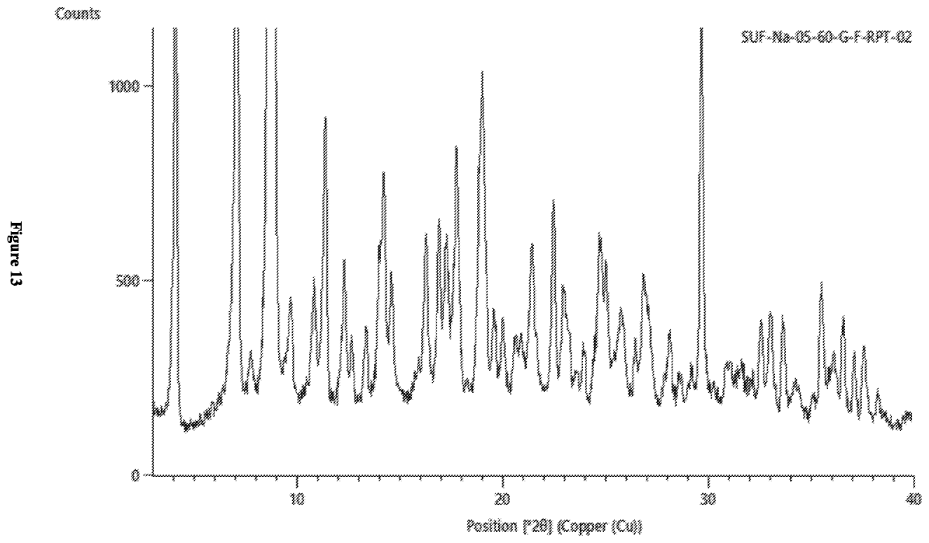

In one embodiment, the present invention provides a solid form of Saflufenacil-sodium (SNa6) having an X-ray diffractogram pattern substantially as shown in FIG. 13.

In one embodiment, the present invention provides a solid form of the Saflufenacil-sodium (SNa6), wherein the solid form exhibits at least 3 of the following peaks expressed as degrees 2θ values in an X-ray diffractogram recorded using Cu—K_{α} a radiation at 25° C.:
peak (1): 2θ=4.1±0.2
peak (2): 2θ=7.1±0.2
peak (3): 2θ=8.7±0.2
peak (4): 2θ=13.4±0.2
peak (5): 2θ=16.9±0.2
peak (6): 2θ=17.2±0.2
peak (7): 2θ=24.7±0.2
   or
   the solid form of the Saflufenacil-sodium (SNa6), has a secondary peak list, wherein the solid form exhibits at least 5 of the following peaks expressed as degrees 2θ values in an X-ray diffractogram recorded using Cu—K_{α} radiation at 25° C.:
peak (1): 2θ=4.1±0.2
peak (2): 2θ=7.1±0.2
peak (3): 2θ=8.7±0.2
peak (4): 2θ=9.7±0.2
peak (5): 2θ=11.4±0.2
peak (6): 2θ=13.4±0.2
peak (7): 2θ=16.9±0.2
peak (8): 2θ=17.2±0.2
peak (9): 2θ=19.0±0.2
peak (10): 2θ=21.4±0.2
peak (11): 2θ=22.5±0.2
peak (12): 2θ=24.7±0.2.

In one embodiment, the present invention provides a solid form of the Saflufenacil-sodium (SNa6), wherein at least 4 of the peaks (1) to (7) or at least 7 of the peaks (1) to (12) are exhibited.

In one embodiment, the present invention provides a solid form of the Saflufenacil-sodium (SNa6), wherein all the peaks (1) to (7) or of the peaks (1) to (12) are exhibited.

In one embodiment, the present invention provides a solid form of the Saflufenacil-sodium (SNa7), wherein the solid form exhibits at least 2 of the following peaks expressed as degrees 2θ values in an X-ray diffractogram recorded using Cu—K_{α} radiation at 25° C.:
peak (1): 2θ=5.7±0.2
peak (2): 2θ=6.8±0.2
peak (3): 2θ=8.7±0.2
peak (4): 2θ=17.6±0.2.

In one embodiment, the present invention provides a solid form of the Saflufenacil-sodium (SNa7), wherein at least 3 of the peaks (1) to (4) are exhibited.

In one embodiment, the present invention provides a solid form of the Saflufenacil-sodium (SNa7), wherein all the peaks (1) to (4) are exhibited.

In one embodiment, the present invention provides a solid form of the Saflufenacil-sodium (SNa7), wherein the solid form exhibits at least 3 of the following peaks expressed as degrees 2θ values in an X-ray diffractogram recorded using Cu—K_{α} radiation at 25° C.:
peak (1): 2θ=5.7±0.2
peak (2): 2θ=6.8±0.2
peak (3): 2θ=8.7±0.2
peak (4): 2θ=11.6±0.2
peak (5): 2θ=13.9±0.2
peak (6): 2θ=17.6±0.2.

In one embodiment, the present invention provides a solid form of the Saflufenacil-sodium (SNa7), wherein at least 3 of the peaks (1) to (6) are exhibited.

In one embodiment, the present invention provides a solid form of the Saflufenacil-sodium (SNa7), wherein all the peaks (1) to (6) are exhibited.

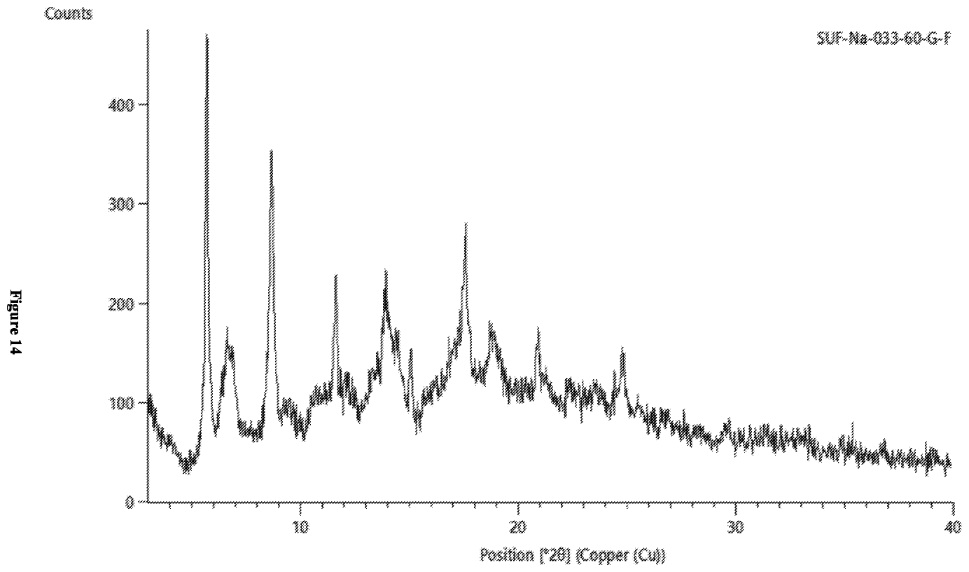

In one embodiment, the present invention provides a solid form of Saflufenacil-sodium (SNa7) having an X-ray diffractogram pattern substantially as shown in FIG. 14.

In one embodiment, the present invention provides a solid form of the Saflufenacil-sodium (SNa7), wherein the solid form exhibits at least 2 of the following peaks expressed as degrees 2θ values in an X-ray diffractogram recorded using Cu—K_{α} radiation at 25° C.:
peak (1): 2θ=5.7±0.2
peak (2): 2θ=6.8±0.2
peak (3): 2θ=8.7±0.2
peak (4): 2θ=17.6±0.2
   or
   the solid form of the Saflufenacil-sodium (SNa7), has a secondary peak list, wherein the solid form exhibits at least 3 of the following peaks expressed as degrees 2θ values in an X-ray diffractogram recorded using Cu—K_{α} radiation at 25° C.:
peak (1): 2θ=5.7±0.2
peak (2): 2θ=6.8±0.2
peak (3): 2θ=8.7±0.2
peak (4): 2θ=11.6±0.2
peak (5): 2θ=13.9±0.2
peak (6): 2θ=17.6±0.2.

In one embodiment, the present invention provides a solid form of the Saflufenacil-sodium (SNa7), wherein at least 3 of the peaks (1) to (4) or at least 3 of the peaks (1) to (6) are exhibited.

In one embodiment, the present invention provides a solid form of the Saflufenacil-sodium (SNa7), wherein all the peaks (1) to (4) or of the peaks (1) to (6) are exhibited.

In one embodiment, the present invention provides a solid form of the Saflufenacil-sodium (SNa8), wherein the solid form exhibits at least 2 of the following peaks expressed as degrees 2θ values in an X-ray diffractogram recorded using Cu—K_{α} radiation at 25° C.:
peak (1): 2θ=6.5±0.2
peak (2): 2θ=12.9±0.2
peak (3): 2θ=13.5±0.2
peak (4): 2θ=18.3±0.2.

In one embodiment, the present invention provides a solid form of the Saflufenacil-sodium (SNa8), wherein at least 3 of the peaks (1) to (4) are exhibited.

In one embodiment, the present invention provides a solid form of the Saflufenacil-sodium (SNa8), wherein all the peaks (1) to (4) are exhibited.

In one embodiment, the present invention provides a solid form of the Saflufenacil-sodium (SNa8), wherein the solid form exhibits at least 3 of the following peaks expressed as degrees 2θ values in an X-ray diffractogram recorded using Cu—K_{α} radiation at 25° C.:
peak (1): 2θ=6.5±0.2
peak (2): 2θ=12.1±0.2
peak (3): 2θ=12.9±0.2
peak (4): 2θ=13.5±0.2
peak (5): 2θ=17.5±0.2
peak (6): 2θ=18.3±0.2
peak (7): 2θ=22.3±0.2.

In one embodiment, the present invention provides a solid form of the Saflufenacil-sodium (SNa8), wherein at least 4 of the peaks (1) to (7) are exhibited.

In one embodiment, the present invention provides a solid form of the Saflufenacil-sodium (SNa8), wherein all the peaks (1) to (7) are exhibited.

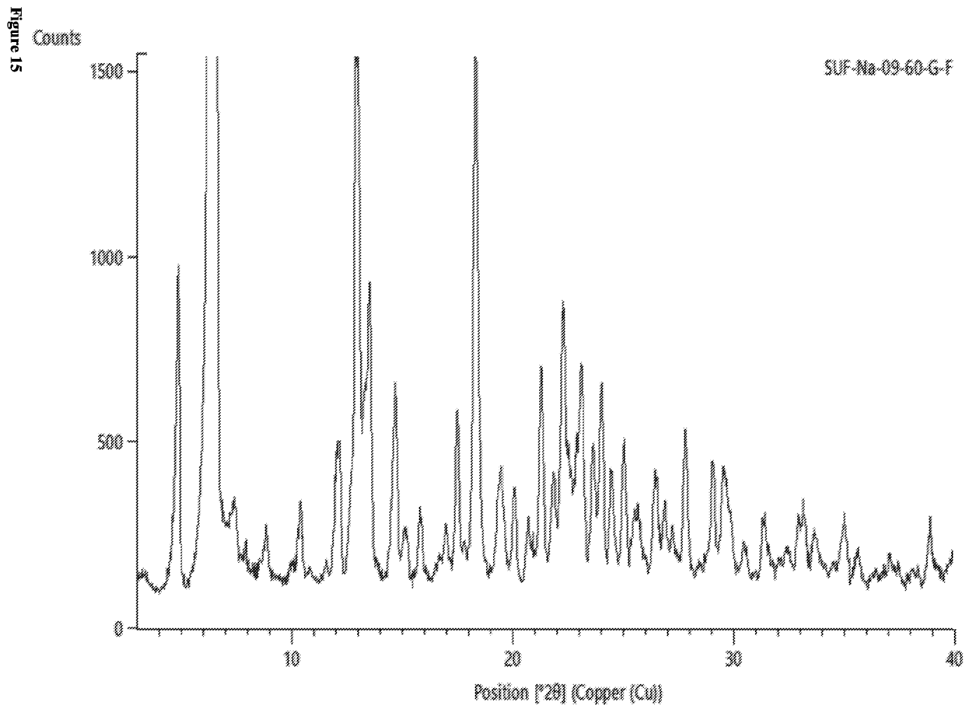

In one embodiment, the present invention provides a solid form of Saflufenacil-sodium (SNa8) having an X-ray diffractogram pattern substantially as shown in FIG. 15.

In one embodiment, the present invention provides a solid form of the Saflufenacil-sodium (SNa8), wherein the solid form exhibits at least 2 of the following peaks expressed as degrees 2θ values in an X-ray diffractogram recorded using Cu—K_{α} radiation at 25° C.:
peak (1): 2θ=6.5±0.2
peak (2): 2θ=12.9±0.2
peak (3): 2θ=13.5±0.2
peak (4): 2θ=18.3±0.2
   or
   the solid form of the Saflufenacil-sodium (SNa8), has a secondary peak list, wherein the solid form exhibits at least 3 of the following peaks expressed as degrees 2θ values in an X-ray diffractogram recorded using Cu—K_{α} radiation at 25° C.:
peak (1): 2θ=6.5±0.2
peak (2): 2θ=12.1±0.2
peak (3): 2θ=12.9±0.2
peak (4): 2θ=13.5±0.2
peak (5): 2θ=17.5±0.2
peak (6): 2θ=18.3±0.2
peak (7): 2θ=22.3±0.2.

In one embodiment, the present invention provides a solid form of the Saflufenacil-sodium (SNa8), wherein at least 3 of the peaks (1) to (4) or at least 4 of the peaks (1) to (7) are exhibited.

In one embodiment, the present invention provides a solid form of the Saflufenacil-sodium (SNa8), wherein all the peaks (1) to (4) or of the peaks (1) to (7) are exhibited.

In one embodiment, the present invention provides a solid form of saflufenacil sodium, wherein the X-ray diffraction pattern is substantially as shown in FIG. 1, 2, 3, 4, 6, 7, 8, 9, 10, 11, 12, 13, 14, or 15.

In one embodiment, the present invention provides a process of preparing the solid form of Saflufenacil-sodium, the process comprising:

i. providing a solution of saflufenacil in an organic solvent;

ii. adding a base;

iii. optionally, heating;

iv. optionally, adding an anti-solvent;

v. optionally, cooling.

The solid obtained by the process of the present invention may be separated from the mixture by the known ways, optionally washed and dried.

In one embodiment, the present invention provides a process of preparing the solid form of Saflufenacil-sodium, wherein the organic solvent comprises any suitable solvent for achieving the desired result, including, without limitation: methanol, ethanol, toluene, chlorobenzene (MCB), 2-methyl-tetrahydrofurane (Me-THF), acetone, ethyl acetate, isopropyl acetate, N,N-dimethylacetamide (DMAC), 2-butanol, dichloromethane, n-heptan, propyl acetate, n-butyl acetate, petroleum ether, n-heptane or methyl isobutyl ketone (MIBK) or any mixture thereof.

In one embodiment, the present invention provides a process of preparing the solid form of Saflufenacil-sodium, wherein the anti-solvent is any organic solvent in which Saflufenacil-sodium has low solubility and can, for example, comprise alkanes like n-hexane or n-heptane.

In one embodiment, the present invention provides a process of preparing the solid form of Saflufenacil-sodium, wherein the base comprises any suitable base for achieving the desired result, including, without limitation: sodium methoxide, sodium ethoxide, sodium isopropoxide, sodium acetate, sodium trifluoroacetate, sodium tert-butoxide, sodium carbonate, or sodium bicarbonate or any mixture thereof.

In one embodiment, the present invention provides a process of preparing the solid form of Saflufenacil-sodium, wherein the temperature of the heating is about 45-about 85° C.

In one embodiment, the present invention provides a process of preparing the solid form of Saflufenacil-sodium, wherein the temperature of the heating is about 30-about 90° C.

In one embodiment, the present invention provides a process of preparing the solid form of Saflufenacil-sodium, wherein the saflufenacil is dissolved in the solvent system at a temperature of from about room temperature to about the reflux temperature of the solution.

In one embodiment, the present invention provides a process of preparing the solid form of Saflufenacil-sodium, wherein the temperature of the cooling is about −10-about +10° C.

In one embodiment, the present invention provides a solid form of Saflufenacil-potassium (SK), wherein the solid form exhibits at least 3 of the following peaks expressed as degrees 2θ values in an X-ray diffractogram recorded using Cu—K_{α} radiation at 25° C.:
peak (1): 2θ=6.2±0.2
peak (2): 2θ=12.5±0.2
peak (3): 2θ=17.4±0.2
peak (4): 2θ=24.4±0.2
peak (5): 2θ=25.5±0.2.

In one embodiment, the present invention provides a solid form of Saflufenacil-potassium (SK), wherein at least 4 of the peaks (1) to (5) are exhibited.

In one embodiment, the present invention provides a solid form of Saflufenacil-potassium (SK), wherein all the peaks (1) to (5) are exhibited.

In one embodiment, the present invention provides a solid form of Saflufenacil-potassium (SK), wherein the solid form exhibits at least 2 of the following peaks expressed as degrees 2θ values in an X-ray diffractogram recorded using Cu—K_{α} radiation at 25° C.:
peak (1): 2θ=4.9±0.2
peak (2): 2θ=10.7±0.2
peak (3): 2θ=14.8±0.2
peak (4): 2θ=20.1±0.2.

In one embodiment, the present invention provides a solid form of Saflufenacil-potassium (SK), wherein at least 3 of the peaks (1) to (4) are exhibited.

In one embodiment, the present invention provides a solid form of Saflufenacil-potassium (SK), wherein all the peaks (1) to (4) are exhibited.

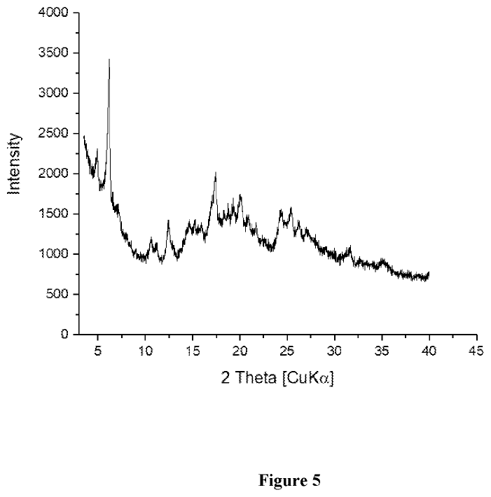

In one embodiment, the present invention provides a solid form of Saflufenacil-potassium (SK), wherein the X-ray diffraction pattern is substantially as shown in FIG. 5.

In one embodiment, the present invention provides a solid form of the Saflufenacil-potassium (SK), wherein the solid form exhibits at least 3 of the following peaks expressed as degrees 2θ values in an X-ray diffractogram recorded using Cu—K_{α} radiation at 25° C.:
peak (1): 2θ=6.2±0.2
peak (2): 2θ=12.5±0.2
peak (3): 2θ=17.4±0.2
peak (4): 2θ=24.4±0.2
peak (5): 2θ=25.5±0.2
   or
   the solid form of the Saflufenacil-potassium (SK), wherein the solid form exhibits at least 2 of the following peaks expressed as degrees 2θ values in an X-ray diffractogram recorded using Cu—K_{α} radiation at 25° C.:
peak (1): 2θ=4.9±0.2
peak (2): 2θ=10.7±0.2
peak (3): 2θ=14.8±0.2
peak (4): 2θ=20.1±0.2.

In one embodiment, the present invention provides a solid form of the Saflufenacil-potassium (SK), wherein at least 4 of the peaks (1) to (5) or at least 3 of the peaks (1) to (4) are exhibited.

In one embodiment, the present invention provides a solid form of the Saflufenacil-potassium (SK), wherein all the peaks (1) to (5) or of the peaks (1) to (4) are exhibited.

In one embodiment, the present invention provides a process of preparing the solid form of Saflufenacil-potassium, the process comprising:

i. providing a solution of saflufenacil in an organic solvent;

ii. adding a base;

iii. optionally, heating;

iv. optionally, adding an anti-solvent;

v. optionally, cooling.

In one embodiment, the present invention provides a process of preparing the solid form of Saflufenacil-potassium, wherein the organic solvent comprises any suitable solvent for achieving the desired result, including, without limitation: toluene, chlorobenzene (MCB), 2-methyl-tetrahydrofurane (Me-THF), acetone, ethyl acetate, isopropyl acetate, N,N-dimethylacetamide (DMAC), or methyl isobutyl ketone (MIBK), or any mixture thereof.

In one embodiment, the present invention provides a process of preparing the solid form of Saflufenacil-potassium, wherein the base comprises and suitable base for achieving the desired result, including, without limitation: potassium methoxide, potassium ethoxide, potassium isopropoxide, potassium trifluoroacetate, potassium tert-butoxide, potassium hydroxide, potassium hydroxide, potassium carbonate, or potassium bicarbonate, or any mixture thereof.

In one embodiment, the present invention provides a process of preparing the solid form of Saflufenacil-potassium, wherein the temperature of the heating is about 30-about 90° C.

In one embodiment, the present invention provides a process of preparing the solid form of Saflufenacil-potassium, wherein the saflufenacil is dissolved in the solvent system at a temperature of from about room temperature to about the reflex temperature of the solution.
In one embodiment, the present invention provides a process of preparing the solid form of Saflufenacil-potassium, further comprising adding an acid to the conversion of the solid forms of Saflufenacil-sodium or Saflufenacil-potassium to Saflufenacil.

In one embodiment, the present invention provides a herbicidal composition comprising one or more of the Saflufenacil-sodium or Saflufenacil-potassium or Saflufenacil-sodium or Saflufenacil-potassium solid forms of Saflufenacil.

The solid form of Saflufenacil-sodium or Saflufenacil-potassium may represent a crystalline form in any of 3 types of particle shapes: needle like particles, rod like particles, plate like particles, and combinations thereof. The particle size may vary from about 50-100, 200-300, 300, 350-400 and 500-550 micron, depending on the shape of the particles.

In one embodiment, the present invention provides an herbicidal composition, wherein the composition is a formulation selected from suspension concentrates (SC), oil-based suspension concentrates (OD), soluble granules (SG), dispersible concentrates (DC), emulsion seed dressings, suspension seed dressings, granules (GR), microgranules (MG), water-dispersible granules (WG), soluble powder (SP), wettable powder (WP) and soluble liquid (SL).

In one embodiment, the present invention provides a herbicidal composition, wherein the composition is a soluble granules (SG).

In one embodiment, the present invention provides a herbicidal composition, wherein the composition is a soluble liquid (SL).

In one embodiment, the present invention provides a herbicidal composition comprising the Saflufenacil-sodium or Saflufenacil-potassium or Saflufenacil-sodium or Saflufenacil-potassium solid forms of Saflufenacil, further comprising one or more additional herbicides.

In yet another aspect, the present invention provides a method of controlling harmful weeds in a field of useful crops, the method comprising applying to the field the Saflufenacil-sodium or Saflufenacil-potassium or Saflufenacil-sodium or Saflufenacil-potassium solid forms of Saflufenacil.

In yet another aspect, the present invention provides a use of the Saflufenacil-sodium or Saflufenacil-potassium or Saflufenacil-sodium or Saflufenacil-potassium solid forms of Saflufenacil in the control of a harmful weed.

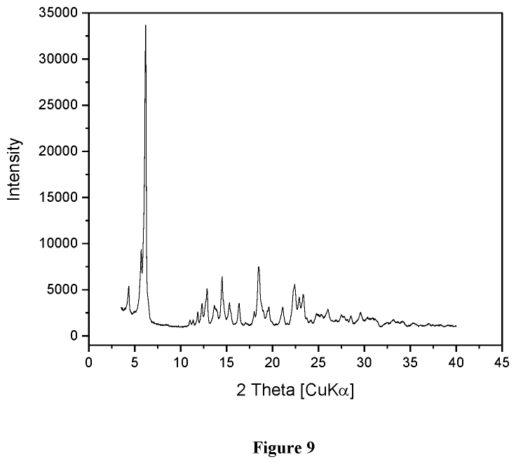

In yet another aspect, the present invention provides a solid form of Saflufenacil-sodium or Saflufenacil-potassium substantially as hereinbefore described, having reference to any of FIGS. 1 to 9.

In yet another aspect, the present invention provides a process of making the solid form of Saflufenacil-sodium or Saflufenacil-potassium substantially as hereinbefore described.

In yet another aspect, the present invention provides a method for controlling harmful weeds substantially as hereinbefore described.

In yet another aspect, the present invention provides a herbicidal composition comprising one or more of the Saflufenacil-sodium or Saflufenacil-potassium or solid form of Saflufenacil-sodium or Saflufenacil-potassium and at least one additional pesticide.

Surprisingly, the solvents used in the preparation of Saflufenacil-sodium may yield different patterns in X-ray analysis of prepared Saflufenacil-sodium solid form or Saflufenacil-potassium solid form. Furthermore, it was surprisingly discovered that freshly obtained Saflufenacil-sodium solid forms, may exhibit low crystallinity properties. During storage stability tests, the Saflufenacil-sodium solid form, may show an increase in crystallinity.

Additional herbicides may be selected from the following list: 2,4-D ester and amine, Aminopyralid, Amitrole, Atrazine, Bixlozone, Carbetamide, Cinmethylin, Clethodim, Clopyralid, Diquat, Diuron, Florasulam, Flumioxazin, Fluroxypyr, Glufosinate, Glyphosate, Haloxyfop,

Imazapic, Imazapyr, Imazethapyr, Indaziflam, Isoxaben, Isoxaflutole, Mesotrione, Metribuzin, Paraquat, Pendimethalin, Picloram, Propaquizafop, Propyzamide, Prosulfocarb, Pyroxasulfone, Simazine, S-metolachlor, Terbuthylazine, Triallate, Triasulfuron, Trifluralin.

Additional pesticides may be selected from herbicides, fungicides and insecticides and fertilizers. In embodiments described herein, the solid form of Saflufenacil-sodium or Saflufenacil-potassium may be prepared by dissolving Saflufenacil in an organic solvent and/or a solvent system comprising one or more mixture or combination of solvents. The Saflufenacil starting material is dissolved in the organic solvent system to form a concentrated solution having a concentration of 0.1% to 50% by weight of the Saflufenacil with or without stirring. The dissolution may be carried out with or without heating. Preferably, the solution is prepared at the temperature of 20° C.-30° C. The concentration of Saflufenacil in the final solution depends on the solubility of Saflufenacil in the organic solvent being employed.

Solvation or dissolution of Saflufenacil may occur by adding the Saflufenacil to the organic solvent or vice versa.

In embodiments, the Saflufenacil may be solid or in a solution.

The resulting reaction mixture may be heterogeneous or homogeneous.

The addition of the base may occur simultaneously or in portions over a set period of time. The base may be added as a concentrate in a solution.

In embodiments, after the addition of the base or the base solution, heat may be applied.

In embodiments, the duration of the reaction to yield the Saflufenacil-sodium or saflufenacil-potassium solid forms is about 0-60 min.

In embodiments, the crystallization of Saflufenacil-sodium or Saflufenacil-potassium may be carried out in an organic solvent selected from chlorobenzene or methyl-tetrahydrofuran and maybe improved by addition of anti-solvent.

In embodiments, the crystallization of Saflufenacil-sodium or Saflufenacil-potassium may be improved by cooling of the reaction mixture to low temperature.

In embodiments, the base used for the synthesis of Saflufenacil-sodium or Saflufenacil-potassium solid forms is sodium methoxide or potassium tert-butoxide.

In an embodiment, an acid may be used to prepare Saflufenacil from the Saflufenacil-sodium or Saflufenacil-potassium solid forms.

In embodiments, the acid may be selected from any acid with the pKa of lower than pKa of Saflufenacil (4.41), meaning any acid with pKa lower than 4.

In an embodiment, the acid is hydrochloric acid.

In one aspect of the invention, crystallization of the solid form of Saflufenacil-sodium or

Saflufenacil-potassium is aided by adding seed crystals of the desired crystalline form during crystallization, which can promote or accelerate the process of crystallization.

In embodiments, it is beneficial to keep the conditions of the reaction dry, to avoid degradation due to moisture.

Thereafter, in embodiments, the separated solid is preferably washed with a suitable solvent, which may be the same solvent system used for the preparation of concentrated solution in step (i) or a different solvent. Washing is usually carried out under cooling, for example between room temperature and 0° C., to reduce the loss of the crystallized product. The washing temperature depends upon the solubility of the crystals in the solvent system being employed.

It was surprisingly discovered, that by using the Saflufenacil-sodium or Saflufenacil-potassium forms described herein, the process of purification of Saflufenacil synthesis is much more efficient and may yield a high purity product. By the conversion to the Saflufenacil-sodium or Saflufenacil-potassium, the impurities of the reaction of the Saflufenacil synthesis, can be separated, then after, the conversion to pure Saflufenacil is easy and efficient.

The solid form of Saflufenacil-sodium or Saflufenacil-potassium of the present invention is particularly suitable for formulating into an herbicidal composition.

Accordingly, in a further aspect, the present invention provides an herbicidal composition comprising the solid form of Saflufenacil-sodium or Saflufenacil-potassium hereinbefore described.

The herbicidal compositions may comprise the solid form of Saflufenacil-sodium or Saflufenacil-potassium in any suitable amount to provide the required activity. In embodiments, preference is given to compositions comprising less than 80% by weight of the solid form of Saflufenacil-sodium or Saflufenacil-potassium, more preferably less than 50% by weight. Compositions comprising about 4% by weight of the solid form of Saflufenacil-sodium or Saflufenacil-potassium are preferred for many applications.

The solid form of Saflufenacil-sodium or Saflufenacil-potassium may be formulated in a known manner to provide a range of customary formulations. Examples of such formulations include suspension concentrates (SC), oil-based suspension concentrates (OD), soluble granules (SG), dispersible concentrates (DC), emulsion seed dressings, suspension seed dressings, granules (GR), microgranules (MG) and water-dispersible granules (WG).

The solid form of Saflufenacil-sodium or Saflufenacil-potassium is particularly suitable for formulation as a soluble granules (SG) or soluble liquid (SL). In addition to the active compound, soluble granules typically comprise surfactants, and, if appropriate, one or more thickeners, antifreeze agents, biocides and/or any other necessary or suitable adjuvants.

The solid form of Saflufenacil-sodium or Saflufenacil-potassium may be present in the soluble granules (SG) composition at a concentration sufficient to achieve the required dosage in the field, for example from about 0.1% to about 90% by weight of the total mixture. In general, the soluble granules (SG) formulations are prepared by extending the solid form of Saflufenacil-sodium or Saflufenacil-potassium with a solvent, in particular water, one or more dispersants or surfactants, and one or more other auxiliaries.

Suitable dispersants are known in the art and are commercially available. Suitable dispersants include, but are not limited to, sodium, calcium and ammonium salts of ligninsulfonates (optionally polyethoxylated); sodium and ammonium salts of maleic anhydride copolymers; sodium salts of condensed phenolsulfonic acid; and naphthalene sulfonate-formaldehyde condensates. Ligninsulfonates, such as sodium ligninsulfonates, are particularly useful for use in the compositions of the invention. Naphthalene sulfonate-formaldehyde condensates, such as naphthalenesulfonic acid polymers with formaldehyde, and their salts, such as sodium salts, are also particularly useful for the compositions of the present invention.

Suitable thickeners for inclusion in the compositions are known in the art and are commercially available. Suitable thickening agents include, but are not limited to, guar gum, pectin, casein, carrageenan, xanthan gum, alginates, methylcellulose, hydroxyethylcellulose, hydroxypropylcellulose, and carboxymethylcellulose. Synthetic thickeners may be used and include derivatives of the aforementioned agents, as well as polyvinyl alcohols, polyacrylamides, polyvinylpyrrolidones, various polyethers, their copolymers as well as polyacrylic acids and their salts. Alkylpolyvinylpyrrolidone is a particularly useful thickener for the compositions of the present invention.

Suitable antifreeze agents for inclusion in the compositions are known in the art and are commercially available. Suitable antifreeze agents include, but are not limited to liquid polyols, for example ethylene glycol, propylene glycol or glycerol. The amount of antifreeze agent present is generally from about 1% to about 20% by weight, in particular from about 5% to about 10% by weight, based on the total weight of the composition.

One or more biocides or preservatives may also be included in the composition according to the invention. Suitable biocides are known in the art and include, without limitation, those based on isothiazolones, for example Proxel® GXL.

The solid form of Saflufenacil-sodium or Saflufenacil-potassium may be the only active ingredient in the pesticidal formulation or may be present in combination with one or more other active compounds, including one or more insecticides, attractants, sterilizing agents, bactericides, acaricides, nematicides, fungicides, growth-regulating substances, herbicides, safeners, fertilizers, or semiochemicals.

Preferred active compounds for use in combination with the solid form of Saflufenacil-sodium or Saflufenacil-potassium include, without limitation, 2,4-D in its various forms (acid, salt, ester), dimethylamine-P, imazethapyr, glyphosate, or any mixture thereof.

The compositions of the present invention comprising the solid form of Saflufenacil-sodium or Saflufenacil-potassium are active in controlling all undesirable weeds which can be controlled using known formulations of Saflufenacil. Undesirable weeds that may be controlled include, for example, but are not limited to broad leaved weeds.

In a yet further aspect, the present invention provides a method for controlling undesirable weeds at root and foliage, the method comprising applying to the locus the solid form of Saflufenacil-sodium or Saflufenacil-potassium hereinbefore described.

In some embodiments, the locus is the root. In some embodiments, the locus is the foliage. In a yet further aspect, the present invention provides a method for controlling undesirable weeds, the method comprising applying to the root or foliage of the weed the solid form of Saflufenacil-sodium or Saflufenacil-potassium hereinbefore described.

Depending on the method of application, solid form of Saflufenacil-sodium or Saflufenacil-potassium or the herbicidal compositions comprising thereof, can additionally be employed in a further number of crop plants to control undesirable weeds. Crops which are suitable, for example, include, without limitation:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus armeniaca, Prunus avium, Prunus cerasus, Prunus dulcis, Prunus domesticua, Prunus persica, Pyrus communis, Ribes sylvestre, Ricinus communis, Saccharum officinarum, Secale cereale, Solanum tuberosum, Sorghum bicolor (S. vulgare), Theobroma cacao, Trifolium pratense, Triticum aestivum, Triticum durum, Vicia faba, Vitis vinifera and Zea mays.

In a still further aspect, the present invention provides the use of the solid form of Saflufenacil-sodium or Saflufenacil-potassium hereinbefore described in the control of a harmful weed infestation.

In embodiments, the formulations described herein can be, but not limited to, solid formulations, including such suspension concentrates and/or granular formulations. The present disclosure contemplates all vehicles by which the synergistic or with additive effect compositions can be formulated for delivery and used as an herbicide.

In a preferred embodiment, the present invention provides a method for purification of Saflufenacil using Saflufenacil-sodium or Saflufenacil-potassium according previously described solid forms of Saflufenacil-sodium or Saflufenacil-potassium.

As will be readily appreciated, any material to which the disclosed compositions can be added may be used, provided they yield the desired utility without significant interference with the activity of these synergistic or additive effect compositions as herbicidal agents.

Wettable powders, which may be compacted to form water-dispersible granules, comprise an intimate mixture of the synergistic or additive effect composition, a carrier and agriculturally acceptable surfactants. The concentration of the disclosed composition in the wettable powder is usually from about 10% to about 90% by weight, more preferably about 25% to about 75% by weight, based on the total weight of the formulation. In the preparation of wettable powder formulations, the synergistic or additive effect composition can be compounded with any of the finely divided solids.

The disclosed compositions may optionally include combinations that can comprise at least 1% by weight of one or more of the compositions with another pesticidal compound. Such additional pesticidal compounds may be fungicides, insecticides, nematocides, miticides, arthropodicides, bactericides or combinations thereof that are compatible with the synergistic or additive effect compositions of the present disclosure in the medium selected for application, and not antagonistic to the activity of the present compounds. Accordingly, in such embodiments the other pesticidal compound is employed as a supplemental toxicant for the same or for a different pesticidal use. The pesticidal compound and the synergistic composition can generally be mixed together in a weight ratio of from 1:100 to 100:1.

Solids may exist in either amorphous or crystalline forms. In the case of crystalline forms, molecules are positioned in 3-dimensional lattice sites.

When a compound recrystallizes from a solution or slurry, it may crystallize with different spatial lattice arrangements, a property referred to as “polymorphism,” With the different crystal forms individually being referred to as a “polymorph”. Different polymorphic forms of a given substance may differ from each other with respect to one or more physical properties, such as solubility and dissociation, true density, crystal shape, compaction behavior, flow properties, and/or solid state stability. Solvates are crystalline solid adducts containing either stoichiometric or nonstoichiometric amounts of a solvent incorporated within the crystal structure. If the incorporated solvent is water, the solvates are also commonly known as hydrates. Solvate or hydrate are also commonly known as “pseudopolymorph”.

New polymorphic, hydrate, or solvate forms can provide various advantages, including improved physical characteristics such as stability or solubility.

The solid forms of Saflufenacil-sodium or Saflufenacil-potassium, disclosed herein, provide improved physical characteristics such as stability or solubility, compared to known polymorph and hydrate of Saflufenacil as well as more efficiency in solid formulations. Therefore, the salts allow for the preparation of a more efficient formulation of Saflufenacil.

Unless defined otherwise, all technical and scientific terms used herein have the meaning commonly understood by persons of ordinary skill in the art to which this subject matter pertains. The compounds and formulations of the invention are useful for controlling weeds and other undesirable plants and pests. “Control” and “controlling” as used herein have the their usual meaning in the agrochemical industry, that is, inter alia, the capacity to kill, prevent growth or reproduction, or to diminish the health of unwanted plants or other pest in a given locus, by interfering in the unwanted plant's or pest's mechanisms, such as metabolism, photosynthesis and/or cell division.

As used herein, “weed” includes any undesired vegetation.

As used herein, the term “effective” or “agriculturally effective” when used in connection with an amount of the compound, combination, mixture, or composition refers to an amount of the compound, combination, mixture, or composition that achieves an agriculturally beneficial level of control and/or prevention of the weed or pest when applied, for example, to the locus where the weed or pest is to be controlled and/or prevented.

As used herein, the term “mixture” or “combination” refers, but is not limited, to a combination in any physical form, e.g., blend, solution or the like.

As used herein, the term “composition” includes a mixture or mixtures of the solid form of the compound of the present invention with another component, including at least one additional herbicide.

As used herein, the term “tank mix” means one or more of the components of the mixture or composition of the present invention and/or one or more of the excipients which are added are mixed in a spray tank at the time of spray application or prior to spray application.

As used herein, the term “agriculturally acceptable carrier” refers to a solvent which is known and accepted in the art for the formation of compositions for agricultural or horticultural use. The term “agriculturally acceptable” can include any carrier or other component that is known and accepted in the art for the formation of or inclusion in compositions for agricultural or horticultural use.

As used herein and as context dictates, the term “additive” refers to any substance that itself is not an active ingredient but is added to the composition. Examples of additives include, but are not limited to, adjuvants, surfactants, emulsifiers, anti-freeze agents, anti-foam agents, and preservatives.

In some embodiments, the compositions and formulations described herein comprise one or more agriculturally acceptable carriers and/or additives. In some embodiments, the compositions and formulations described herein do not comprise a carrier or additive.

As used herein, the term “excipient” refers to any chemical which has no pesticidal activity, such as surfactant(s), solvent(s), or adjuvant(s). One or more excipients can be added to any mixture or composition disclosed herein.

The term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one of skill in the art, that in some specific instances, an embodiment can be described using the language “consisting essentially of” or “consisting of.”

The term “about” herein specifically includes ±10% from the indicated values in the range. In addition, the endpoints of all ranges directed to the same component or property herein are inclusive of the endpoints, are independently combinable, and include all intermediate points and ranges.

It is understood that where a parameter range is provided, all integers within that range, and tenth thereof, are also provided by the invention. For example, “70 to 80° C.” includes 70° C., 70.1° C., 70.2° C., 70.3° C. etc. up to 80° C.

In yet another embodiment, the product of any of the disclosed processes can be isolated from the reaction mixture by any conventional techniques well-known in the art. Such isolation techniques can include, without limitation, one or more of the following: concentration, extraction, precipitation, cooling, filtration, crystallization, and centrifugation, followed by drying.

In yet another embodiment, the product of any of the disclosed processes can be optionally purified by any conventional techniques well-known in the art. Such purification techniques may include, without limitation, one or more of the following: precipitation, crystallization, slurrying, washing in a suitable solvent, filtration through a packed-bed column, dissolution in an appropriate solvent, and re-precipitation by addition of a second solvent in which the compound is insoluble, or any combination thereof.

It is suggested that the slight difference between the solid forms of Saflufenacil-sodium, which is observed in XRD, may be caused due to different states of crystallinity as well as a function of the solvate created and the ratios of the solvents in the crystal. Also, it is suggested that the crystalline form may be dependent on the temperature and various storage conditions.

Each embodiment disclosed herein is contemplated as being applicable to each of the other disclosed embodiments, thus, all combinations of the various elements described herein are within the scope of the invention. The elements recited in solid form embodiments can be used in the process, composition, method and use embodiments described herein and vice versa.

While the present subject matter has been shown and described with reference to preferred embodiments thereof, it will be understood by those skilled in the art that many alternatives, modifications and variations may be made thereto without departing from the spirit and scope thereof. Accordingly, it is intended to embrace all such alternatives, modifications, and variations that fall within the spirit and broad scope of the appended claims.

All publications, patents and patent applications referred to in this specification are herein incorporated in their entirety by reference, to the same extent as if each individual publication, patent or patent application was specifically and individually indicated to be incorporated herein by reference.

The following examples illustrate the practice of the present subject matter in some of its embodiments but should not be construed as limiting the scope of the present subject matter. Other embodiments will be apparent to one skilled in the art from consideration of the specification and examples. It is intended that the specification, including the examples, is considered exemplary only, without limiting the scope and spirit of the present subject matter.

### EXAMPLES

The solid forms of Saflufenacil-sodium and Saflufenacil-potassium were prepared according to the present invention, as shown, but not limited to below, are analyzed, characterized and differentiated by X-ray powder diffraction. Another suitable technique to analyze, characterize and differentiate the individual forms is by Raman and/or IR spectroscopy.

The XRD samples were analyzed by the X-ray Diffractometer, produced by Empyrean, Panalytical tube Cu, voltage—40 kV, current—30 mA.

### Example 1

### Preparation of Saflufenacil

To a clean and dry 4 neck flask equipped with a mechanical stirrer, thermocouple, condenser and additional funnel were charged 500 ml of chlorobenzene, 50 g (97%, 0.1 mol) of immediate intermediate of formula Ib (a precursor to Saflufenacil),

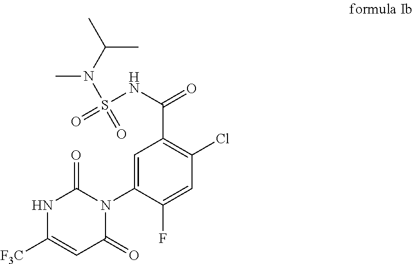 [SMILES: CC(C)N(C)S(=O)(=O)NC(=O)c1cc(-n2c(=O)cc(C(F)(F)F)[nH]c2=O)c(F)cc1Cl]

3.3 g (0.01 mol) TBAB and 43 g (0.5 mol) of NaHCO₃ at 25-30° C. under good stirring.
To this mixture at the same temperature during half an hour were fed 15.2 g (0.12 mol) of DMS over the period of 30 min. at 25-30° C. Reaction mass was heated to 60-65° C. and stirred at this temperature during about 3 h up to the end of reaction (intermediate of formula Ib less than 2 area % according HPLC).

Reaction mass was cooled to 55° C. and 250 mL of 8% aqueous HCl solution were fed to the reaction mass over the period of 25-30 min at 55-60° C. Stirring was stopped and the layers were separated at 55-60° C. for 5-10 min.

Bottom organic layer (chlorobenzene layer, containing the product) was washed twice at the same temperature with 250 mL of water. If the pH of the last washing water is lower than 6, third washing should be done.

About 250 mL of chlorobenzene were distilled out from the reaction mass at 60-65° C. under vacuum.

To the received solution 50 mL of methanol were added and the mixture was cooled to 25° C.

Amount of the product in prepared solution was checked and equivalent amount 16 g (0.09 mol) of 30% MeONa in methanol were added over the period of 30 min at 25-30° C.

The mixture was stirred at 25-30° C. for about an hour and 500 ml of n-heptane were added to the solution over the period of 25-30 min at 25-30° C. Reaction mass was stirred about 6 h at this temperature, cooled to 5° C. and mixed about 2 h at 5-10° C.

Saflufenacil-sodium was filtered at 5-10° C. and washed on the filter with 150 mL of chilled n-heptane.

To another flask were charged 250 mL of 8% aqueous HCl solution and under good stirring wet cake of Saflufenacil-sodium was added portion wise at 25-30° C.

Reaction mixture was stirred at 25-30° C. for 1 h and filtered. Saflufenacil was washed on the filter with 100 mL of water at 25-30° C. and dried under the vacuum at 45-50° C. 38 g of the product with purity 99% were prepared. Yield 73%.

### Example 2

### Preparation of Solid Form of Saflufenacil-Sodium, Using MCB-40 g Preparation

51 g of Saflufenacil 98% purity (0.1 mol) were dissolved in 550 mL of mono-chlorobenzene (MCB) and in order to reduce amount of water to minimum about 200 mL of MCB were distilled out under the vacuum keeping temperature of the mixture at 60-65° C. At this temperature 50 mL of methanol were added and the mixture was cooled to 25° C. At this temperature 17.5 g of 30% sodium methoxide solution in methanol (0.097 mol) were fed under good stirring to the mixture during about 0.5 h.

Resulting mixture was stirred additional 40 min at 25-30° C. and 500 mL of n-heptane were added during 1 h at the same temperature. The reaction mass was stirred at 25-30° C. overnight, cooled to 10° C. and mixed additional 1 h at 5-10° C.

Precipitated product was filtered at 5-10° C., washed with 150 mL of n-heptane and dried under the vacuum at room temperature during 3 h. 38.2 g of dry Saflufenacil sodium were prepared.

The crystals were characterized by powder X-ray and determined to be the solid form of Saflufenacil-sodium salt as shown in FIGS. 1 and 2.

The preparation of an 80 g sample was preformed exactly the same way as described in Example 2 with double the quantities.

The crystals were characterized by powder X-ray and determined to be the solid form of Saflufenacil-sodium salt as shown in FIG. 6.

### Example 3

### Preparation of Solid Form of Saflufenacil-Sodium, Using 2-Me-THF-40 g Preparation

51 g of Saflufenacil 98% purity (0.1 mol) were dissolved in 350 mL of 2-methytetrahydrofurane (Me-THF) and in order to reduce amount of water to minimum about 150 mL of Me-THF were distilled out under atmospheric pressure. Prepared solution was cooled to 25° C. At this temperature 17.5 g of 30% sodium methoxide solution in methanol (0.097 mol) were fed under good stirring to the mixture during about 0.5 h.

Resulting mixture was stirred additional 40 min at 25-30° C. and 450 mL of n-heptane were added during 1.5 hat the same temperature. The reaction mass was stirred at 25-30° C. overnight, and precipitated product was filtered at 25-30° C. The product on the filter was washed with 150 mL of the mixture n-heptane/Me-THF 1:1 and dried under the vacuum at room temperature during 3 h. 30.7 g of dry Saflufenacil sodium were prepared.

The crystals were characterized by powder X-ray and determined to be the solid form of Saflufenacil-sodium salt as shown in FIGS. 3 and 4.

The preparation of an 80 g sample was performed exactly the same way as described in Example 2, with double the quantities.

The crystals were characterized by powder X-ray and determined to be the solid form of Saflufenacil-sodium salt as shown in FIG. 7.

### Example 4

### Preparation of Solid Form of Saflufenacil-Sodium, Using MCB-10 kg Preparation

To a 250 liter GL reactor equipped with a mechanical stirrer, thermocouple, condenser and connected to the scrubber were charged 170 kg of chlorobenzene, 15 kg (99%, 30.6 mol) of intermediate of formula Ib (a precursor to Saflufenacil),

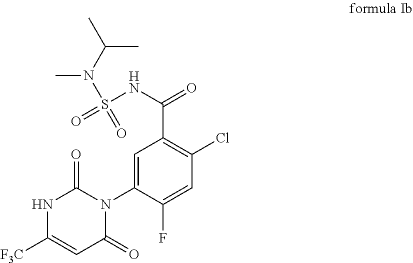 [SMILES: CC(C)N(C)S(=O)(=O)NC(=O)c1cc(-n2c(=O)cc(C(F)(F)F)[nH]c2=O)c(F)cc1Cl]

975 g TBAB and 12.6 kg (150 mol) of NaHCO₃ at 25-30° C. under good stirring.

To this mixture at the same temperature during half an hour were fed 4.5 kg (35.2 mol) of DMS over the period of 30 min. at 25-30° C. Reaction mass was heated to 60-65° C. and stirred at this temperature during about 3 h up to the end of reaction (intermediate of formula Ib less than 2 area % according HPLC).

Reaction mass was cooled to 55° C. and 86.6 kg of 6.4% aqueous HCl solution were fed to the reaction mass over the period of 25-30 min and mixture was heated to 75-80° C. Stirring was stopped and the layers were separated at this temperature during 30 min.

Bottom organic layer (chlorobenzene layer, containing the product) was washed twice at the same temperature with 75 kg of water. If the pH of the last washing water is lower than 6, third washing should be done.

About 80 kg of chlorobenzene were distilled out from the reaction mass at 75-80° C. under vacuum.

To the received solution 12 kg of methanol were added and the mixture was cooled to 25° C.

Amount of the product in prepared solution was checked and equivalent amount 4.8 kg (26.7 mol) of 30% MeONa in methanol were added over the period of 1 hour at 25-30° C. To the mixture at the same temperature 101 kg of n-heptane were added over the period of 25-30 min at 25-30° C. Reaction mass was stirred about 6 h at this temperature, cooled to 5° C. and mixed about 2 h at 5-10° C.

Saflufenacil-sodium was filtered at 5-10° C. and washed twice on the filter with 30 kg of chilled n-heptane.

### Example 5

### Preparation of Saflufenacil Na Salt for the Following Examples 6-11

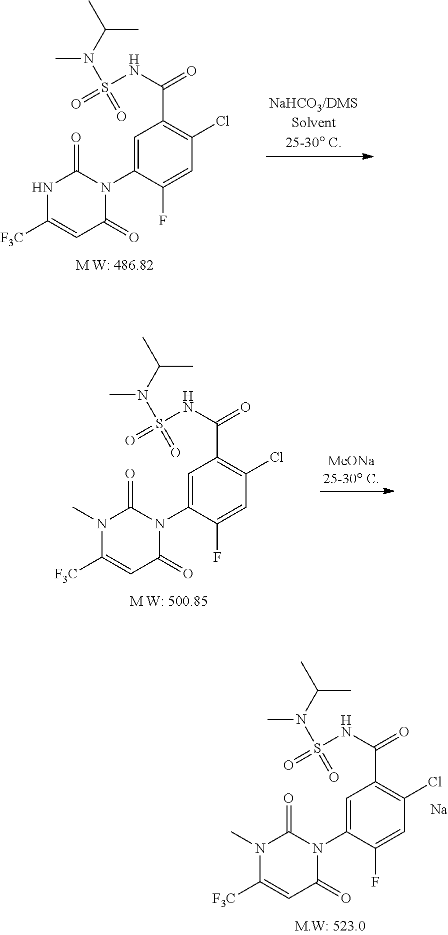

A clean and dry 4 neck round bottom flask (RBF) equipped with a mechanical stirrer, thermocouple, condenser, and additional funnel, was charged with 1000 mL of Chlorobenzene. A 2-chloro-5-(2,6-dioxo-4-(trifluoromethyl)-3,6-dihydropyrimidin-1(2H)-yl)-4-fluoro-N-(N-isopropyl-N-methylsulfamoyl)benzamide (100 g, 0.207 mol) at 25-30° C. was charged, followed by addition of Tetra butyl ammonium bromide (6.75 g, 0.0209 mol). Then added slowly Sodium bicarbonate 86.4 g (1.02 mol) to the above reaction mixture at 25-30° C. Finally, Dimethyl sulfate (30.49g, 0.241 mol) was added over the period of 25-30 min. at 25-30° C., then the reaction mass was heated to 60-65° C. The reaction mass was maintained for 2 h at 50-55° C. The reaction was monitored using HPLC. After the completion of the reaction 500 mL 25% Aqueous Hydrochloric acid was added to the reaction mass over the period of 25-30 min at 25-30° C. Then the reaction mass was heated to 55-60° C. to get a clear solution for 25-30 min. The layers were allowed to separate at 55-60° C., the bottom organic layer was collected into the RBF followed by 500 mL of water washing of the organic layer at 55-60° C. (the pH was ensured to not be acidic (<6)). Then the chlorobenzene was distilled up to 500 mL (5V) at 65-70° C. (the moisture of the reaction mass was ensured to be less than <0.2). Finally, a solution of MeONa in methanol was added to form the sodium salt. n-Heptane (10V) was added to the solution to precipitate the Na salt. The salt was filtered and washed the bed with n-Heptane (3V). Finally the sodium salt was dried to get 85 g of sodium salt with 97.8% HPLC purity.
This sodium salt of saflufenacil was taken for the preparation of examples 6 to 11.

### Example 6

### Preparation of Solid Form of Saflufenacil-Sodium, Using Different Volumes of MeOH/Chlorobenzene Mixture and MeOH/2-Methyltetrahydrofuran Mixture to Obtain Form SNa3

### SUF-Na-08(2V)-052(8V)-27-G-S

50 mg of saflufenacil Na salt and 0.1 mL/0.4 mL (2V/8V) of MeOH/Chlorobenzene mixture were stirred at RT for 1.5 hour and a slurry mixture was obtained. Then the mixture was heated to 50° C. and stirred at 850 rpm for 1 hour to get a clear solution. n-Heptane (7 mL) was added into the solution and stirred for 10 minutes at 50° C. The precipitate was centrifuged at 7000 rpm for 5 minutes and an off-white solid was analyzed by XRPD without any further drying to obtain form SNa3.

### SUF-Na-08(3V)-052(2V)-27-G-S

50 mg of saflufenacil Na salt and 0.15 mL/0.1 mL (3V/2V) of MeOH/Chlorobenzene mixture were stirred at RT for 1.5 hour and a slurry mixture was obtained. Then the mixture was heated to 50° C. and stirred at 850 rpm for 1 hour to get a clear solution. n-Heptane (7 mL) was added into the solution and stirred for 10 minutes at 50° C. The precipitate was centrifuged at 7000 rpm for 5 minutes and an off-white solid was analyzed by XRPD without any further drying to obtain form SNa3.

### SUF-Na-08(4V)-039(1V)-27-G-S

50 mg of saflufenacil Na salt and 0.2 mL /0.05 mL (4V/1V) of MeOH/2-Methyltetrahydrofuran mixture were stirred at RT for 1.5 hour and a slurry mixture was obtained. Then the mixture was heated to 50° C. and stirred at 850 rpm for 1 hour to get a clear solution. n-Heptane (14 mL) was added into the solution and stirred for 10 minutes at 50° C. The precipitate was centrifuged at 7000 rpm for 5 minutes and an off-white solid was analyzed by XRPD without any further drying to obtain form SNa3.

### SUF-Na-08(4V)-052(1V)-27-G-S

50 mg of saflufenacil Na salt and 0.2 mL/0.05 mL (4V/1V) of MeOH/Chlorobenzene mixture were stirred at RT for 1.5 hour and a slurry mixture was obtained. Then the mixture was heated to 50° C. and stirred at 850 rpm for 1 hour to get a clear solution. n-Heptane (14 mL) was added into the solution and stirred for 10 minutes at 50° C. The precipitate was centrifuged at 7000 rpm for 5 minutes and an off-white solid was analyzed by XRPD without any further drying to obtain form SNa3.

### SUF-Na-08(8V)-052(2V)-57-G-S-HEPT

50 mg of saflufenacil Na salt and 0.4 mL/0.1 mL (8V/2V) of MeOH/Chlorobenzene mixture were stirred at RT and 850 rpm for 1 hour to get a clear solution. n-Heptane (15 mL) was added into the solution and stirred for 15 minutes at RT. The precipitate was centrifuged at 7000 rpm for 5 minutes and an off-white solid was analyzed by XRPD without any further drying to obtain form SNa3.

### SUF-Na-08(9V)-039(1V)-57-G-S-HEPT

50 mg of saflufenacil Na salt and 0.45 mL/0.05 mL (9V/1V) of MeOH/2-Methyltetrahydrofuran mixture were stirred at RT and 850 rpm for 1 hour to get a clear solution. n-Heptane (15 mL) was added into the solution and stirred for 25 minutes at RT. The precipitate was centrifuged at 7000 rpm for 5 minutes and an off-white solid was analyzed by XRPD without any further drying to obtain form SNa3.

### SUF-Na-08(9V)-052(1V)-57-G-S-HEPT

50 mg of saflufenacil Na salt and 0.45 mL/0.05 mL (9V/1V) of MeOH/Chlorobenzene mixture were stirred at RT and 850 rpm for 1 hour to get a clear solution. n-Heptane (15 mL) was added into the solution and stirred for 15 minutes at RT. The precipitate was centrifuged at 7000 rpm for 5 minutes and an off-white solid was analyzed by XRPD without any further drying to obtain form SNa3.

### Example 7

### Preparation of Solid Form of Saflufenacil-Sodium, Using Different Solvents to Obtain Form SNa4

exp. SUF-Na-013-60-G-F

A slurry mixture of saflufenacil Na salt (50 mg) and 0.5 mL of 2-Butanol was stirred at RT and 850 rpm for 18 hours. The precipitate was centrifuged at 7000 rpm for 5 minutes and an off-white solid was analyzed by XRPD without any further drying to obtain form SNa4.

Exp. SUF-Na-051-60-G-F

A slurry mixture of saflufenacil Na salt (50 mg) and 0.5 mL of Dichloromethane was stirred at RT and 850 rpm for 18 hours. The precipitate was centrifuged at 7000 rpm for 5 minutes and an off-white solid was analyzed by XRPD without any further drying to obtain form SNa4.

Exp. SUF-Na-055-57-G-S-SL-RT-1 hr 45 mins
50 mg of saflufenacil Na salt and 0.15 mL of Dimethylacetamide were stirred at RT and 1000 rpm for 25 min to get a clear solution. n-Heptane (15 mL) was added into the solution and stirred for 1 hour and 45 minutes at RT. The precipitate was centrifuged at 7000 rpm for 5 minutes and an off-white solid was analyzed by XRPD without any further drying to obtain form SNa4.
Exp. SUF-Na-013-60-G-F
A slurry mixture of saflufenacil Na salt (50 mg) and 0.5 mL of 2-Butanol was stirred at RT and 850 rpm for 18 hours. The precipitate was centrifuged at 7000 rpm for 5 minutes and an off-white solid was analyzed by XRPD without any further drying to obtain form SNa4.

### Example 8

### Preparation of Solid Form of Saflufenacil-Sodium, Using Different Solvents to Obtain Form SNa5.Exp. SUF-Na-04-60-G-F

A slurry mixture of saflufenacil Na salt (50 mg) and 0.5 mL of Methyl isobutyl ketone was stirred at RT and 850 rpm for 18 hours. The precipitate was centrifuged at 7000 rpm for 5 minutes and an off-white solid was analyzed by XRPD without any further drying to obtain form SNa5.
Exp. SUF-Na-022-60-G-F
A slurry mixture of saflufenacil Na salt (50 mg) and 0.5 mL of Ethyl acetate was stirred at RT and 850 rpm for 18 hours. The precipitate was centrifuged at 7000 rpm for 5 minutes and an off-white solid was analyzed by XRPD without any further drying to obtain form SNa5.
Exp. SUF-Na-024-60-G-F
A slurry mixture of saflufenacil Na salt (50 mg) and 0.5 mL of Isopropyl acetate was stirred at RT and 850 rpm for 18 hours. The precipitate was centrifuged at 7000 rpm for 5 minutes and an off-white solid was analyzed by XRPD without any further drying to obtain form SNa5.
Exp. SUF-Na-025-60-G-F
A slurry mixture of saflufenacil Na salt (50 mg) and 0.5 mL of Propyl acetate was stirred at RT and 850 rpm for 18 hours. The precipitate was centrifuged at 7000 rpm for 5 minutes and an off-white solid was analyzed by XRPD without any further drying to obtain form SNa5.
Exp. SUF-Na-027-60-G-F
A slurry mixture of saflufenacil Na salt (50 mg) and 0.5 mL of n-Butyl acetate was stirred at RT and 850 rpm for 18 hours. The precipitate was centrifuged at 7000 rpm for 5 minutes and an off-white solid was analyzed by XRPD without any further drying to obtain form SNa5.
Exp. SUF-Na-052-60-G-F
A slurry mixture of saflufenacil Na salt (50 mg) and 0.5 mL of Chlorobenzene was stirred at RT and 850 rpm for 18 hours. The precipitate was centrifuged at 7000 rpm for 5 minutes and an off-white solid was analyzed by XRPD without any further drying to obtain form SNa5.

### Example 9

### Preparation of Solid Form of Saflufenacil-Sodium, Using Methyl Isopropyl Ketone to Obtain Form SNa5

Exp. SUF-Na-05-60-G-F-RPT
A slurry mixture of saflufenacil Na salt (500 mg) and 5 mL of Methyl isopropyl ketone was stirred at RT and 850 rpm for 18 hours. The precipitate was centrifuged at 7000 rpm for 5 minutes and an off-white solid was analyzed by XRPD without any further drying to obtain form SNa6.

### Example 10

### Preparation of Solid Form of Saflufenacil-Sodium, Using Different Solvents to Obtain Form SNa7

Exp. SUF-Na-033-60-G-F
A slurry mixture of saflufenacil Na salt (50 mg) and 0.5 mL of Methyl cyclohexane was stirred at RT and 850 rpm for 18 hours. The precipitate was centrifuged at 7000 rpm for 5 minutes and an off-white solid was analyzed by XRPD without any further drying to obtain form SNa7.
Exp. SUF-Na-046-60-G-F
A slurry mixture of saflufenacil Na salt (50 mg) and 0.5 mL of Petroleum ether was stirred at RT and 850 rpm for 18 hours. The precipitate was centrifuged at 7000 rpm for 5 minutes and an off-white solid was analyzed by XRPD without any further drying to obtain form SNa7.
Exp. SUF-Na-028-60-G-F-RPT-02
A slurry mixture of saflufenacil Na salt (600 mg) and 6 mL of n-Heptane was stirred at RT and 850 rpm for 18 hours. The precipitate was centrifuged at 7000 rpm for 5 minutes and then dried under vacuum (760 mmHg) at RT for 3.5 hours. An off-white solid was analyzed by XRPD without any further drying to obtain form SNa7.

### Example 11

### Preparation of Solid Form of Saflufenacil-Sodium, Using Ethanol to Obtain Form SNa8

A slurry mixture of saflufenacil Na salt (50 mg) and 0.5 mL of ethanol was stirred at RT and 850 rpm for 18 hours. The precipitate was centrifuged at 7000 rpm for 5 minutes and an off-white solid was analyzed by XRPD without any further drying to obtain form SNa8.

### Example 12

### Determination of the Sodium in the Saflufenacil-Sodium Samples

Na ions concentration was checked by ion chromatography and was found 4.2±0.2%.
The saflufenacil sodium salt, is a mono sodium cation containing molecule, as possible from the chemical structure.

### Example 13

### Preparation of Solid Form of Saflufenacil-Potassium

51 g of Saflufenacil 98% purity (0.1 mol) were dissolved in 550 mL of mono-chlorobenzene (MCB) and in order to reduce amount of water to minimum about 200 mL of MCB were distilled out under the vacuum keeping temperature of the mixture at 60-65° C. At this temperature 100 mL of tert-butanol were added and the mixture was cooled to 25° C. At this temperature 110 g of 10% potassium tert-butoxide solution in tert-butanol (0.097 mol) were fed under good stirring to the mixture during about 0.5 h.

Resulting mixture was stirred additional 60 min at 25-30° C. and 500 mL of n-heptane were added during 1 h at the same temperature. The reaction mass was stirred at 25-30° C. overnight, cooled to 10° C. and mixed additional 1 h at 5-10° C.

Precipitated product was filtered at 5-10° C., washed with 150 mL of n-heptane and dried under the vacuum at room temperature during 3 h. 36.2 g of dry Saflufenacil potassium were prepared.

The crystals were characterized by powder X-ray and determined to be the solid form of Saflufenacil-potassium salt as shown in FIG. 5.

### Example 14

### Saflufenacil Formulation and its Preparation—2,4-D and Saflufenacil SG Formulation

| 2,4-D 800/Saflufenacil 50 SG | g/kg |
|---|---|
| 2,4-D Na (96%) | 916.3 |
| Saflufenacil (96%) Na (88.6%) | 56.43 |
| Genapol ® X 080 With Granulation Water | 2 |
| Sucrose | 4.82 |
| Trisodium Phosphate | 20.0 |
| Silfoam ® 150 SP | 0.5 |
| SUM | 1000 |

### Preparation Instructions:

1. Mix together the powders: Saflufenacil Na, 2,4-D Na, Sucrose, Trisodium Phosphate, Silfoam® 150 SP for about 30 min.

2. Mill the powders mixtures using hammer mill.

3. Spray on the mixture granulation water (about 24% based on dry weight) that contains Genapol® X 080 and knead the mixture using Schugi® apparatus, for example.

4. Pass the dough-like material trough extrusion granulator to form short, worm-like granules.

5. Dry the granules using hot-air fluid-bed facility.

| | | | | | Quantity |
|---|---|---|---|---|---|
| Ingredient | Description | Cas | Quantity | Units | Percent |
| 2_4-D_SODIUM_SALT | 2 4-D_SODIUM_SALT | 2702-72-9 | 799.97 | KG | 91.53 |
| GENAPOL_X_080 | Genapol ® X 080 | 78330-21-9 | 2 | KG | 0.2 |
| SAFLUFENACIL_SODIUM | SAFLUFENACIL_SODIUM | | 50 | KG | 5.643 |
| SILFOAM_SP_150 | Silfoam ® SP 150 | N/A | 0.5 | KG | 0.05 |
| SUCROSE | Sucrose | 57-50-1 | 5.77 | KG | 0.577 |
| TRISODIUM_PHOSPHATE_{—} | TRISODIUM_PHOSPHATE | 7601-54-9 | 20 | KG | 2 |

### Example 15

### Saflufenacil Formulation and its Preparation—Saflufenacil SP Formulation

| Ingredient | Quantity (g/kg) |
|---|---|
| 2_4-D_SODIUM_SALT | 799.97 |
| SAFLUFENACIL_SODIUM | 50 |
| SILFOAM ®_SP_150 | 0.5 |
| SUCROSE | 129.5 |
| TRISODIUM_PHOSPHATE_{—} | 20 |

### Preparation Instructions:

1. Mix together the powders for about 30 min.

2. Mill the powders mixtures using hammer mill.

In addition, any priority document(s) of this application is/are hereby incorporated herein by reference in its/their entirety.

What is claimed is:

1. Saflufenacil-sodium or Saflufenacil-potassium.

2. The Saflufenacil-sodium or Saflufenacil-potassium of claim 1, wherein the Saflufenacil-sodium is a salt or the Saflufenacil-potassium is a salt.

3. The salt of claim 2, wherein the salt is a sodium or potassium salt of Saflufenacil.

4. A solid form of the Saflufenacil-sodium or of the Saflufenacil-potassium of any of claims 1-3.

5. A solid form of the Saflufenacil-sodium or Saflufenacil-potassium of any of claims 1-4.

6. The solid form of the Saflufenacil-sodium or Saflufenacil-potassium of any of claims 1-5, wherein the solid form is an anhydrous form, a crystalline form, a hydrate form, a solvate form, a polymorph form, a crystalline form with low crystallinity or a crystalline form with high crystallinity, or any combination thereof.

7. The solid form of the Saflufenacil-sodium (SNa1) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=5.9±0.3
peak (2): 2θ=14.7±0.2
peak (3): 2θ=18.6±0.2
peak (4): 2θ=19.5±0.2
peak (5): 2θ=22.7±0.4
or
the solid form of the Saflufenacil-sodium (SNa1) of any of claims 1-6, has a secondary pick list, wherein the solid form exhibits at least 3 of the following peaks expressed as degrees 2θ values in an X-ray diffractogram recorded using Cu—K_{α} radiation at 25° C.:
peak (1): 2θ=6.1±0.2
peak (2): 2θ=14.9±0.2
peak (3): 2θ=18.5±0.2
peak (4): 2θ=19.7±0.2
peak (5): 2θ=23.3±0.2.

8. The solid form of the Saflufenacil-sodium (SNa1) of claim 7, wherein at least 3 of the peaks (1) to (5) are exhibited.

9. The solid form of the Saflufenacil-sodium (SNa1) of claim 7, wherein all the peaks (1) to (5) are exhibited.

10. The solid form of the Saflufenacil-sodium (SNa1a) of any of claims 1-9, wherein the solid form exhibits at least 2 of the following peaks expressed as degrees 2θ values in an X-ray diffractogram recorded using Cu—K_{α} radiation at 25° C.:
peak (1): 2θ=4.3±0.2
peak (2): 2θ=16.4±0.2
peak (3): 2θ=19.6±0.2
peak (4): 2θ=22.5±0.2.

11. The solid form of the Saflufenacil-sodium (SNa1a) of claim 10, wherein at least 3 of the peaks (1) to (4) are exhibited.

12. The solid form of the Saflufenacil-sodium (SNa1a) of claim 10, wherein all the peaks (1) to (4) are exhibited.

13. The solid form of the Saflufenacil-sodium (SNa1b) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=6.1±0.2
peak (2): 2θ=14.7±0.2
peak (3): 2θ=18.3±0.2
peak (4): 2θ=19.5±0.2
peak (5): 2θ=23.1±0.2
or
the solid form of the Saflufenacil-sodium (SNa1b) of any of claims 1-6, has a secondary pick list, wherein the solid form exhibits at least 2 of the following peaks expressed as degrees 2θ values in an X-ray diffractogram recorded using Cu—K_{α} radiation at 25° C.:
peak (1): 2θ=16.3±0.2
peak (2): 2θ=19.5±0.2
peak (3): 2θ=22.5±0.2.

14. The solid form of the Saflufenacil-sodium (SNa1b) of claim 13, wherein at least 4 of the peaks (1) to (5) are exhibited.

15. The solid form of the Saflufenacil-sodium (SNa1b) of claim 13, wherein all the peaks (1) to (5) the peaks (1) to (3) are exhibited.

16. The solid form of the Saflufenacil-sodium (SNa2) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=5.9±0.2
peak (2): 2θ=14.9±0.2
peak (3): 2θ=18.5±0.2
peak (4): 2θ=22.7±0.2
or
the solid form of the Saflufenacil-sodium (SNa2a) of any of claims 1-6, has a secondary peak list, wherein the solid form exhibits at least 2 of the following peaks expressed as degrees 2θ values in an X-ray diffractogram recorded using Cu—K_{α} radiation at 25° C.:
peak (1): 2θ=4.9±0.2
peak (2): 2θ=12.3±0.2
peak (3): 2θ=16.3±0.2
peak (4): 2θ=19.5±0.2.

17. The solid form of the Saflufenacil-sodium (SNa2) of claim 16, wherein at least 3 of the peaks (1) to (4) are exhibited.

18. The solid form of the Saflufenacil-sodium (SNa2) of claim 16, wherein all the peaks (1) to (4) are exhibited.

19. The solid form of the Saflufenacil-sodium (SNa2b) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=5.9±0.2
peak (2): 2θ=14.7±0.2
peak (3): 2θ=18.5±0.2
peak (4): 2θ=22.7±0.2
or
the solid form of the Saflufenacil-sodium (SNa2b) of any of claims 1-6, has a secondary peak list, wherein the solid form exhibits at least 2 of the following peaks expressed as degrees 2θ values in an X-ray diffractogram recorded using Cu—K_{α} radiation at 25° C.:
peak (1): 2θ=4.9±0.2
peak (2): 2θ=12.1±0.2
peak (3): 2θ=16.5±0.2
peak (4): 2θ=19.6±0.2.

20. The solid form of the Saflufenacil-sodium (SNa2b) of claim 19, wherein at least 3 of the peaks (1) to (4) are exhibited.

21. The solid form of the Saflufenacil-sodium (SNa2b) of claim 19, wherein all the peaks (1) to (4) are exhibited.

22. The solid form of any of the Saflufenacil-sodium (SNa1c) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=6.2±0.2
peak (2): 2θ=14.5±0.2
peak (3): 2θ=18.5±0.2
peak (4): 2θ=19.6±0.2
peak (5): 2θ=22.4±0.2
or
the solid form of any of the Saflufenacil-sodium (SNa1c) of claims 1-6, has a secondary peak list, wherein the solid form exhibits at least 2 of the following peaks expressed as degrees 2θ values in an X-ray diffractogram recorded using Cu—K_{α} radiation at 25° C.:
peak (1): 2θ=11.0±0.2
peak (2): 2θ=11.4±0.2
peak (3): 2θ=11.8±0.2
peak (4): 2θ=13.7±0.2.

23. The solid form of the Saflufenacil-sodium (SNa1c) of claim 22, wherein at least 3 of the peaks (1) to (5) or of the peaks (1) to (4) are exhibited.

24. The solid form of the Saflufenacil-sodium (SNa1c) of claim 22, wherein all the peaks (1) to (5) or of the peaks (1) to (4) are exhibited.

25. The solid form of the Saflufenacil-sodium (SNa3) of any of claims 1-6, wherein the solid form exhibits at least 2 of the following peaks expressed as degrees 2θ values in an X-ray diffractogram recorded using Cu—K_{α} radiation at 25° C.:
peak (1): 2θ=5.8±0.2
peak (2): 2θ=11.7±0.2
peak (3): 2θ=17.7±0.2
peak (4): 2θ=23.7±0.2
or
the solid form of the Saflufenacil-sodium (SNa3) of any of claims 1-6, has a secondary peak list, wherein the solid form exhibits at least 4 of the following peaks expressed as degrees 2θ values in an X-ray diffractogram recorded using Cu—K_{α} radiation at 25° C.:
peak (1): 2θ=5.8±0.2
peak (2): 2θ=11.7±0.2
peak (3): 2θ=15.2±0.2
peak (4): 2θ=17.7±0.2
peak (5): 2θ=21.0±0.2
peak (6): 2θ=23.7±0.2
peak (7): 2θ=24.9±0.2
peak (8): 2θ=29.8±0.2.

26. The solid form of the Saflufenacil-sodium (SNa3) of claim 25, wherein at least 3 of the peaks (1) to (4) or at least 5 of the peaks (1) to (8) are exhibited.

27. The solid form of the Saflufenacil-sodium (SNa3) of claim 25, wherein all the peaks (1) to (4) or of the peaks (1) to (8) are exhibited.

28. The solid form of the Saflufenacil-sodium (SNa4) of any of claims 1-6, wherein the solid form exhibits at least 2 of the following peaks expressed as degrees 2θ values in an X-ray diffractogram recorded using Cu—K_{α} radiation at 25° C.:
peak (1): 2θ=6.7±0.2
peak (2): 2θ=13.4±0.2
peak (3): 2θ=20.1±0.2
or
the solid form of the Saflufenacil-sodium (SNa4) of any of claims 1-6, has a secondary peak list, wherein the solid form exhibits at least 4 of the following peaks expressed as degrees 2θ values in an X-ray diffractogram recorded using Cu—K_{α} radiation at 25° C.:
peak (1): 2θ=6.7±0.2
peak (2): 2θ=13.4±0.2
peak (3): 2θ=14.2±0.2
peak (4): 2θ=18.0±0.2
peak (5): 2θ=20.1±0.2
peak (6): 2θ=23.1±0.2
peak (7): 2θ=24.0±0.2
peak (8): 2θ=24.5±0.2.

29. The solid form of the Saflufenacil-sodium (SNa4) of claim 28, wherein at least 5 of the peaks (1) to (8) are exhibited.

30. The solid form of the Saflufenacil-sodium (SNa4) of claim 28, wherein all the peaks (1) to (3) or of the peaks (1) to (8) are exhibited.

31. The solid form of the Saflufenacil-sodium (SNa5) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=4.2±0.2
peak (2): 2θ=7.1±0.2
peak (3): 2θ=13.5±0.2
peak (4): 2θ=17.4±0.2
peak (5): 2θ=24.9±0.2
peak (6): 2θ=26.0±0.2
or
the solid form of the Saflufenacil-sodium (SNa5) of any of claims 1-6, has a secondary peak list, wherein the solid form exhibits at least 4 of the following peaks expressed as degrees 2θ values in an X-ray diffractogram recorded using Cu—K_{α} radiation at 25° C.:
peak (1): 2θ=4.2±0.2
peak (2): 2θ=7.1±0.2
peak (3): 2θ=9.8±0.2
peak (4): 2θ=13.5±0.2
peak (5): 2θ=14.3±0.2
peak (6): 2θ=17.4±0.2
peak (7): 2θ=21.5±0.2
peak (8): 2θ=24.9±0.2
peak (9): 2θ=26.0±0.2.

32. The solid form of the Saflufenacil-sodium (SNa5) of claim 31, wherein at least 4 of the peaks (1) to (6) or at least 6 of the peaks (1) to (9) are exhibited.

33. The solid form of the Saflufenacil-sodium (SNa5) of claim 31, wherein all the peaks (1) to (6) or of the peaks (1) to (9) are exhibited.

34. The solid form of the Saflufenacil-sodium (SNa6) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=4.1±0.2
peak (2): 2θ=7.1±0.2
peak (3): 2θ=8.7±0.2
peak (4): 2θ=13.4±0.2
peak (5): 2θ=16.9±0.2
peak (6): 2θ=17.2±0.2
peak (7): 2θ=24.7±0.2
or
the solid form of the Saflufenacil-sodium (SNa6) of any of claims 1-6, has a secondary peak list, wherein the solid form exhibits at least 5 of the following peaks expressed as degrees 2θ values in an X-ray diffractogram recorded using Cu—K_{α} radiation at 25° C.:
peak (1): 2θ=4.1±0.2
peak (2): 2θ=7.1±0.2
peak (3): 2θ=8.7±0.2
peak (4): 2θ=9.7±0.2
peak (5): 2θ=11.4±0.2
peak (6): 2θ=13.4±0.2
peak (7): 2θ=16.9±0.2
peak (8): 2θ=17.2±0.2
peak (9): 2θ=19.0±0.2
peak (10): 2θ=21.4±0.2
peak (11): 2θ=22.5±0.2
peak (12): 2θ=24.7±0.2.

35. The solid form of the Saflufenacil-sodium (SNa6) of claim 34, wherein at least 4 of the peaks (1) to (7) or at least 7 of the peaks (1) to (12) are exhibited.

36. The solid form of the Saflufenacil-sodium (SNa6) of claim 34, wherein all the peaks (1) to (7) or of the peaks (1) to (12) are exhibited.

37. The solid form of the Saflufenacil-sodium (SNa7) of any of claims 1-6, wherein the solid form exhibits at least 2 of the following peaks expressed as degrees 2θ values in an X-ray diffractogram recorded using Cu—K_{α} radiation at 25° C.:
peak (1): 2θ=5.7±0.2
peak (2): 2θ=6.8±0.2
peak (3): 2θ=8.7±0.2
peak (4): 2θ=17.6±0.2
or
the solid form of the Saflufenacil-sodium (SNa7) of any of claims 1-6, has a secondary peak list, wherein the solid form exhibits at least 3 of the following peaks expressed as degrees 2θ values in an X-ray diffractogram recorded using Cu—K_{α} radiation at 25° C.:
peak (1): 2θ=5.7±0.2
peak (2): 2θ=6.8±0.2
peak (3): 2θ=8.7±0.2
peak (4): 2θ=11.6±0.2
peak (5): 2θ=13.9±0.2
peak (6): 2θ=17.6±0.2.

38. The solid form of the Saflufenacil-sodium (SNa7) of claim 37, wherein at least 3 of the peaks (1) to (4) or at least 3 of the peaks (1) to (6) are exhibited.

39. The solid form of the Saflufenacil-sodium (SNa7) of claim 37, wherein all the peaks (1) to (4) or of the peaks (1) to (6) are exhibited.

40. The solid form of the Saflufenacil-sodium (SNa8) of any of claims 1-6, wherein the solid form exhibits at least 2 of the following peaks expressed as degrees 2θ values in an X-ray diffractogram recorded using Cu—K_{α} radiation at 25° C.:
peak (1): 2θ=6.5±0.2
peak (2): 2θ=12.9±0.2
peak (3): 2θ=13.5±0.2
peak (4): 2θ=18.3±0.2
or
the solid form of the Saflufenacil-sodium (SNa8) of any of claims 1-6, has a secondary peak list, wherein the solid form exhibits at least 3 of the following peaks expressed as degrees 2θ values in an X-ray diffractogram recorded using Cu—K_{α} radiation at 25° C.:
peak (1): 2θ=6.5±0.2
peak (2): 2θ=12.1±0.2
peak (3): 2θ=12.9±0.2
peak (4): 2θ=13.5±0.2
peak (5): 2θ=17.5±0.2
peak (6): 2θ=18.3±0.2
peak (7): 2θ=22.3±0.2.

41. The solid form of the Saflufenacil-sodium (SNa8) of claim 40, wherein at least 3 of the peaks (1) to (4) or at least 4 of the peaks (1) to (7) are exhibited.

42. The solid form of the Saflufenacil-sodium (SNa8) of claim 40, wherein all the peaks (1) to (4) or of the peaks (1) to (7) are exhibited.

43. The solid form of Saflufenacil-sodium of any of claims 1-42, wherein the X-ray diffraction pattern is substantially as shown in FIG. 1, 2, 3, 4, 6, 7, 8, 9, 10, 11, 12, 13, 14 or 15.

44. A process of preparing the solid form of the Saflufenacil-sodium of any of claims 1-43, the process comprising:
i. providing a solution of saflufenacil in an organic solvent;
ii. adding a base;
iii. optionally, heating;
iv. optionally, adding an anti-solvent;
v. optionally, cooling.

45. The process of claim 44, wherein the organic solvent comprises: methanol, ethanol, toluene, chlorobenzene (MCB), 2-methyl-tetrahydrofurane (Me-THF), acetone, ethyl acetate, isopropyl acetate, N,N-dimethylacetamide (DMAC), 2-butanol, dichloromethane, n-heptan, propyl acetate, n-butyl acetate, petroleum ether, n-heptane or methyl isobutyl ketone (MIBK) or any mixture thereof.

46. The process of any of claims 44-45, wherein the base comprises: sodium methoxide, sodium ethoxide, sodium isopropoxide, sodium acetate, sodium trifluoroacetate, sodium tert-butoxide, sodium carbonate, or sodium bicarbonate or any mixture thereof.

47. The process of any of claims 44-46, wherein the temperature of the heating is about 45-about 85° C.

48. The process of any of claims 44-47, wherein the temperature of the heating is about 30-about 90° C.

49. The process of any of claims 44-48, wherein the Saflufenacil is dissolved in the solvent system at a temperature of from about room temperature to about the reflux temperature of the solution.

50. The solid form of the Saflufenacil-potassium (SK) of any of claims 1-6, wherein the solid form exhibits at least 3 of the following peaks expressed as degrees 2θ values in an X-ray diffractogram recorded using Cu—K_{α} radiation at 25° C.:
peak (1): 2θ=6.2±0.2
peak (2): 2θ=12.5±0.2
peak (3): 2θ=17.4±0.2
peak (4): 2θ=24.4±0.2
peak (5): 2θ=25.5±0.2
or
the solid form of the Saflufenacil-potassium (SK) of any of claims 1-6, wherein the solid form exhibits at least 2 of the following peaks expressed as degrees 2θ values in an X-ray diffractogram recorded using Cu—K_{α} radiation at 25° C.:
peak (1): 2θ=4.9±0.2
peak (2): 2θ=10.7±0.2
peak (3): 2θ=14.8±0.2
peak (4): 2θ=20.1±0.2.

51. The solid form of the Saflufenacil-potassium (SK) of claim 50, wherein at least 4 of the peaks (1) to (5) or at least 3 of the peaks (1) to (4) are exhibited.

52. The solid form of the Saflufenacil-potassium (SK) of claim 50, wherein all the peaks (1) to (5) or of the peaks (1) to (4) are exhibited.

53. The solid form of the Saflufenacil-potassium of any of claim 1-6 or 50-52, wherein the X-ray diffraction pattern is substantially as shown in FIG. 5.

54. A process of preparing the solid form of the Saflufenacil-potassium (SK) of any of claim 1-6 or 50-53, the process comprising:
i. providing a solution of saflufenacil in an organic solvent;
ii. adding a base;
iii. optionally, heating;
iv. optionally, adding an anti-solvent;
v. optionally, cooling.

55. The process of claim 54, wherein the organic solvent comprises: toluene, chlorobenzene (MCB), 2-methyl-tetrahydrofurane (Me-THF), acetone, ethyl acetate, isopropyl acetate, N,N-dimethylacetamide (DMAC), or methyl isobutyl ketone (MIBK), or any mixture thereof.

56. The process of any of claims 54-55, wherein the base comprises: potassium methoxide, potassium ethoxide, potassium isopropoxide, potassium trifluoroacetate, potassium tert-butoxide, potassium hydroxide, potassium hydroxide, potassium carbonate, or potassium bicarbonate, or any mixture thereof.

57. The process of any of claims 54-56, wherein the temperature of the heating is about 30-about 90° C.

58. The process one of any of claims 54-57, wherein the Saflufenacil is dissolved in the solvent system at a temperature of from about room temperature to about the reflex temperature of the solution.

59. The process of any of claim 44-48 or 54-58, further comprising adding an acid to the conversion of the solid forms of Saflufenacil-sodium or Saflufenacil-potassium to saflufenacil.

60. An herbicidal composition comprising one or more of the saflufenacil-sodium or saflufenacil-potassium or Saflufenacil-sodium or Saflufenacil-potassium solid forms of Saflufenacil of any of claims 1-43 and 50-53.

61. The herbicidal composition of claim 60, wherein the composition is a formulation selected from suspension concentrates (SC), oil-based suspension concentrates (OD), soluble granules (SG), dispersible concentrates (DC), emulsion seed dressings, suspension seed dressings, granules (GR), microgranules (MG), water-dispersible granules (WG), soluble powder (SP), wettable powder (WP) and soluble liquid (SL).

62. The herbicidal composition of any of claim 60 or 61, wherein the composition is a soluble granules (SG).

63. The herbicidal composition of any of claims 60-62, further comprising one or more additional herbicides.

64. A method of controlling harmful weeds in a field of useful crops, the method comprising applying to the field the Saflufenacil-sodium or Saflufenacil-potassium or Saflufenacil-sodium or Saflufenacil-potassium solid forms of Saflufenacil of any of claims 1-43 and 50-53 or the composition of any of claims 60-63.

65. Use of the Saflufenacil-sodium or Saflufenacil-potassium or Saflufenacil-sodium or Saflufenacil-potassium solid forms of Saflufenacil of any of claims 1-43 and 50-53 or the composition of any of claims 60-63 in the control of a harmful weed.

66. A solid form of Saflufenacil-sodium or Saflufenacil-potassium substantially as hereinbefore described, having reference to any of FIGS. 1 to 15.

67. A process of making the solid form of saflufenacil-sodium or Saflufenacil-potassium substantially as hereinbefore described.

68. A method for controlling harmful weeds substantially as hereinbefore described.

69. An herbicidal composition comprising one or more of the Saflufenacil-sodium or Saflufenacil-potassium or solid form of Saflufenacil-sodium or Saflufenacil-potassium according to any one of claims 1-43 and 50-53 or the composition of any of claims 60-63 and at least one additional pesticide.

70. A method for purification of Saflufenacil using the Saflufenacil-sodium or Saflufenacil-potassium of any of claims 1-43 and 50-53.
